(19) United States
(12) Patent Application Publication
(10) Pub. No.: US 2021/0252069 A1
(43) Pub. Date: Aug. 19, 2021

(54) CHIMERIC ANTIGEN RECEPTOR FOR SOLID CANCER AND T CELLS EXPRESSING CHIMERIC ANTIGEN RECEPTOR

(71) Applicant: Seogkyoung Kong, Seoul (KR)

(72) Inventor: Seogkyoung Kong, Seoul (KR)

(21) Appl. No.: 17/274,062

(22) PCT Filed: Sep. 5, 2019
(86) PCT No.: PCT/KR2019/011516

§ 371 Date: Mar. 5, 2021

Related U.S. Application Data
(60) Provisional application No. 62/727,254, filed on Sep. 5, 2018.

(51) Int. Cl. A61K 35/17; C07K 14/54; C07K 14/715; C07K 14/725

(52) U.S. Cl. CPC A61K 35/17; C07K 14/54; A61K 38/00; C07K 14/7051; C07K 14/7155

(57) ABSTRACT

Disclosed is a chimeric antigen receptor with improved persistency.

### CROSS REFERENCE TO RELATED APPLICATIONS

This application is the National Stage of International Application No. PCT/KR2019/011516, filed on Sep. 5, 2019, which claims priority to U.S. Provisional Patent Application No. 62/727,254, filed on Sep. 5, 2018. The contents of both applications are hereby incorporated by reference in their entirety.

### TECHNICAL FIELD

The present invention relates to a novel chimeric antigen receptor (CAR) for effectively treating blood cancer or solid cancer, and to a CAR-T cell in which a chimeric antigen receptor targeting a specific cancer antigen is expressed. In addition, the present invention relates to a vector which expresses a novel CAR for effectively treating blood cancer or solid cancer.

More specifically, the present invention relates to a chimeric antigen receptor comprising an antigen binding domain; a hinge region; a transmembrane domain; a costimulatory domain; and a cytoplasmic signaling domain, in which any one immunoreceptor tyrosine-based activation motif (hereinafter described as “ITAM”) present within a CD3ζ domain, which is a cytoplasmic signaling domain, is mutated for excellent in vivo persistence and anti-tumor effects of CAR-T cells, and to a CAR-T cell in which the chimeric antigen receptor is expressed.

Further, in order to induce a cytokine signal in an antigen-dependent manner during stimulation of cancer antigens, a cleaved cytoplasmic domain of interleukin-7 receptor-α (IL7Rα) or interleukin-2 receptor-β (IL-2Rβ) in addition to the CD3ζ signaling domain and a 4-1BB costimulatory domain was added to a CAR gene so as to reduce the differentiation and exhaustion of CAR-T cells caused by CAR tonic signaling of CAR-T cells, thereby making it capable of exhibiting excellent in vivo persistence and anti-tumor effects.

In addition, in the present invention, a chimeric switch receptor was introduced to convert the immunosuppressive signal in the hostile tumor microenvironment into an activation signal in CAR-T cells.

In addition, in the present invention, the cytokine IL-21 is released out of CAR-T cells so as to aid the activation of innate immune-related cells and increase the content of less-differentiated memory CAR-T cells.

The chimeric antigen receptor according to the present invention can show excellent in vivo persistence and anti-tumor effects of CAR-T cells in a hostile tumor microenvironment, and can simultaneously reduce toxicity, thereby reducing side effects in the patient while improving the treatment effect. In addition, it is possible to expand the scope of use of the present invention by preparing a chimeric antigen receptor-provided allogenic (non-self-derived) CAR-T treatment according to the present invention.

### BACKGROUND

T cells expressing chimeric antigen receptor (CAR) (CAR-T cells) refers to a type of T cell with a recombinant gene, in which a gene encoding a receptor that recognizes cancer cell surface antigens specifically expressed on the surface of cancer cells is introduced into a T cell to kill cancer cells.

Dr. Zelig Eshhar, an Israeli chemist and immunologist at the Weizmann Institute of Science, et al. previously proposed a hypothesis that when T cells are artificially made with a receptor that binds to an antigen specifically expressed in a cancer cell, it is possible to target only cancer cells and trigger an immune response to kill cancer cells. They succeeded in preparing T cells provided with a chimeric antigen receptor, and this was reported in PNAS in 1989.

In the CAR-T cells prepared in the early days (i.e., the first-generation CAR-T cells), only CD3ζ was used as a signaling domain. However, these CAR-T cells had drawbacks in that the treatment effect was insignificant and that the persistence was also short. Therefore, efforts have been made to improve the reactivity of CAR-T cells, and as a result, second-generation CAR-T cells were prepared in which a costimulatory domain (CD28 or CD137/4-1BB) and CD3ζ were combined, and these CAR-T cells showed an increase in the number of CAR-T cells remaining in the body compared to the first-generation CAR-T cells.

In the second-generation CAR-T cells, only one kind of costimulatory domain was used, and CAR-T cells using two kinds of costimulatory domains are referred to as third-generation CAR-T.

With regard to the method of treating cancer using CAR-T cells, three late-stage chronic lymphoid leukemia (CCL) patients were injected with cytotoxic T cells which were modified to recognize CD19. As a result, leukemia was completely cured in two of the patients, and there were reports that the conditions lasted for about 10 months (N Engl J Med 2011, 365:725-733, Aug. 25, 2011; Sic Transl Med 2011 Aug. 10, 3(95):95ra73). In particular, the CAR-T used corresponded to the second-generation CAR-T cells, in which 4-1BB was used as the costimulatory domain and CD3ζ was used as the signaling domain. The antigen binding domain of the CAR-T cells recognizes CD19 found on the surface of leukemia cancer cells as an antigen.

In addition, there was a report that when CTL019 was administered to patients with acute leukemia, 27 out of 30 patients experienced complete remission, 67% of the patients experienced complete remission for over 2 years, and 78% of the patients survived for two years. Considering that the target patients above were relapsed or refractory patients, these results were very surprising (N Engl J Med 2014, 371:1507-1517, Oct. 16, 2014).

Currently, clinical trials for various blood cancers (e.g., lymphoma, myeloma, etc.) are in progress with regard to therapeutic methods using various CAR-T cells, and CAR-T, as a pharmaceutical drug that can be used for the treatment of blood cancer, has entered the market.

However, there are several obstacles to using CAR-T cells for the treatment of solid cancer. For example, when CAR-T cells are administered intravenously to a cancer patient, it is difficult for CAR-T cells to migrate to the tumor site; and in a hostile tumor microenvironment, CAR-T cells are functionally inhibited while simultaneously exhibiting limited persistence and proliferation.

Therefore, there is a need to develop CAR-T cells which can treat solid cancer by overcoming these problems.

### DETAILED DESCRIPTION OF THE INVENTION

### Technical Problem

An object of the present invention is to provide CAR platforms, CAR-T cells, and CAR expression vectors which have remarkably excellent therapeutic effects in solid cancer compared to the previously known CAR-T cells.

The CAR-T cell therapeutic of the present invention for treating solid cancer comprises: (1) a mutated ITAM introduced into the cytoplasmic signaling domain for excellent in vivo persistence and antitumor effects of CAR-T cells; and/or (2) CAR tonic signaling controlled by adding a cleaved cytoplasmic domain of IL7Rα or IL-2Rβ in addition to a CD3ζ signaling domain and a 4-1BB costimulatory domain to a CAR gene so as to induce a cytokine signal in an antigen-dependent manner; and/or (3) a chimeric switch receptor introduced so as to convert the immunosuppressive signal in the hostile tumor microenvironment into an activation signal in CAR-T cells; and/or (4) cytokine IL-21 allowed to release out of CAR-T cells so as to aid the activation of innate immune-related cells and increase the content of less-differentiated memory CAR-T cells.

Due to CAR tonic signaling, CAR-T cells promote anergy, differentiation, exhaustion, and activation-induced CAR-T cell death. In order to increase the therapeutic efficacy of CAR-T cells for treating solid cancer, excellent in vivo persistence and anti-tumor effects of CAR-T cells are essential. Therefore, it is necessary to control the CAR tonic signaling system in strategies for solid cancer treatment.

Since the CAR tonic signaling can be controlled by substitution of a cytosolic signaling domain of CAR-T cell, an antigen-dependent cytokine signal was added into CAR-T cell. Additionally, in order to control the CAR tonic signaling that may occur due to uncontrolled excessive ITAM-based signaling within the CD3ζ domain, one mutated ITAM was introduced into the cytoplasmic signaling domain. By controlling CAR tonic signaling, the excellent in vivo persistence and anti-tumor effects of CAR-T cells were made to last longer.

In addition, a chimeric switch receptor was introduced, which converts the inhibitory signal of TGF-β (i.e., an immunosuppressive cytokine overexpressed in a hostile tumor microenvironment) into an activation signal in CAR-T cells. In addition, it was prepared such that cytokine IL-21 could be released out of CAR-T cells so as to aid the activation of innate immune-related cells and increase the content of less-differentiated memory CAR-T cells.

The chimeric antigen receptor of the present invention can be used to prepare CAR-T cell therapeutics for various carcinomas by changing the cancer antigen target in the antigen binding domain.

### Technical Solution

To solve the problems described above, the present invention provides a polypeptide comprising a chimeric antigen receptor, which comprises an antigen binding domain; a hinge region; a transmembrane domain; a costimulatory domain; and a signaling domain. In particular, the costimulatory domain may comprise a 4-1BB domain or CD28 domain or both a 4-1BB domain and a CD28 domain, in which the signaling domain comprises a CD3ζ domain.

In the present invention, the polypeptide comprises IL-7Rα or a part thereof interposed between the 4-1BB domain and the CD3ζ domain. In particular, a part of IL-7Rα may be a sequence of SEQ ID NO: 15, and a part of IL-2Rβ may be a sequence of SEQ ID NO: 17. A part of the cytoplasmic domain of the IL-7Rα (SEQ ID NO: 15) is a sequence between the 266^{th} and the 328^{th} positions in the entire sequence of IL-7Rα of SEQ ID NO: 14, and a part of the cytoplasmic domain of the IL-2Rβ (SEQ ID NO: 17) is a sequence between the 266^{th} and the 369^{th} positions in the entire sequence of IL-2Rβ of SEQ ID NO: 16.

For example, in vivo persistence and anti-tumor effects of CAR-T cells were increased by controlling the CAR tonic signaling by linking a cleaved cytoplasmic domain of IL-7Rα (amino acids at positions from 265 to 328; SEQ ID NO: 15) or cleaved cytoplasmic domain of IL2Rβ (amino acids at positions from 266 to 369; SEQ ID NO: 17) between 4-1BB (SEQ ID NO: 11) as a costimulatory domain and mutant CD3ζ (in which the positions from 91 to 113 of SEQ ID NO: 18 are substituted with either SEQ ID NO: 31 or SEQ ID NO: 32), in a CAR which is specific to IL-13Ra2, based on the known anti-IL13Ra2 CAR-T cells (YYB-103 of SEQ ID NO: 22: see PCT International Publication No. WO 2017/023138).

In the present invention, three immunoreceptor tyrosine-based activation motifs (ITAMs) are included within the CD3ζ domain, and in a third ITAM region among these, it is preferred that a first motif, YxxL, and a second motif, YxxL, be substituted (in which x and y each represent any amino acid) (see FIG. 3).

Generally, the ITAM within the CD3ζ domain includes two forms of YxxL (or isoleucine (I)) containing tyrosine (Y), which is spaced apart from leucine (L) or isoleucine (I) by a distance of a two amino acid sequence. Since the YxxL/I forms are usually present to be spaced apart by 6 to 8 amino acids, the ITAM can be represented by the structure of YxxL/Ix₍₆₋₈₎YxxL/I.

In the present invention, three ITAMs (positions 21 to 35; positions 60 to 75; and positions 91 to 105 of SEQ ID NO: 18) are included within the CD3ζ domain, which is represented by SEQ ID NO: 18, in which in the CD3ζ domain, it is preferred that in a third ITAM region, a first motif, YxxL (positions 91 to 94 of SEQ ID NO: 18), be substituted with YxxQ, and a second motif region, YxxLHM (positions 102 to 107 of SEQ ID NO: 18), be substituted with YxYVTM; or in a third ITAM region, a first motif, YxxL (positions 91 to 94 of SEQ ID NO: 18), be substituted with YyyL, and a second motif, YxxL (positions 102 to 105 of SEQ ID NO: 18), be substituted with YxxQ (in which x and y each represent any amino acid).

For example, in the present invention, the region including a third ITAM region of the CD3ζ domain may be mutated to a sequence of SEQ ID NO: 31 or 32.

Specifically, a chimeric antigen receptor (CAR) can be used, in which among the three ITAM motifs present within the CD3ζ domain (see SEQ ID NO: 18) (i.e., a known signaling domain of anti-IL13Ra2 CAR-T cells (YYB-103)), the sequence of a third ITAM motif (i.e., positions 91 to 106 of SEQ ID NO: 18) is mutated from “YQGLSTATKDTYDALHMQALPPR” to “YLPQSTATKDTYDYVTMQALPPR” (SEQ ID NO: 31); or among the three ITAM motifs present within the CD3ζ domain (see SEQ ID NO: 18) (i.e., a known signaling domain of anti-IL13Ra2 CAR-T cells (YYB-103)), the sequence of a third ITAM motif (i.e., positions 91 to 105 of SEQ ID NO: 18) is mutated from “YQGLSTATKDTYDALHMQALPPR” to “YLSLSTATKDTYLPQHMQALPPR” (SEQ ID NO: 32) (see FIG. 3).

In particular, in the present invention, it is preferred that in a third ITAM region of the CD3ζ domain, a first motif, YxxL (positions 91 to 94 of SEQ ID NO: 18), be substituted with YxxQ, and a second motif, YxxLHM (positions 102 to 106 of SEQ ID NO: 18), be substituted with YxYVTM; or in a third ITAM region of the CD3ζ domain, a first motif, YxxL (positions 91 to 94 of SEQ ID NO: 18), be substituted with YyyL, and a second motif, YxxL (positions 102 to 105 of SEQ ID NO: 18), be substituted with YxxQ (in which x and y each represent any amino acid).

The polypeptide of the present invention may further contain a cytokine. For example, the polypeptide of the present invention may contain IL-21 as a cytokine to be added. In particular, it is preferred that the cytokine be linked to a chimeric antigen receptor by a self-cleaving peptide.

As the self-cleaving peptide, for example, the known 2A peptides of SEQ ID NOS: 40 to 43 may be used. The self-cleaving peptide undergoes a post-translational cleavage, in which the peptide bond between a proline (P) and a glycine (G) in the C-terminus is digested by cleavage. Therefore, the proteins located upstream and downstream of the self-cleaving peptide are expressed independently of each other.

Accordingly, when the polypeptide according to the present invention is expressed in a T cell, the cytokine can be separated from a chimeric antigen receptor (CAR), and the separated cytokine can be released to the outside of the T cell. Innate immune-related cells can be activated by the separated cytokine (e.g., IL-21), and thereby the content of less-differentiated CAR-T cells can be increased (see FIG. 5).

That is, in vitro differentiation is prevented due to the IL-21 expression during the process of CAR-T cell preparation, which results in an increase of the content of less-differentiated memory CAR-T cells, thereby making it possible to provide excellent in vivo persistence and enhanced anti-tumor effects of CAR-T cells, and which additionally results in activation of the innate immune cells around the solid cancer, thereby making it possible to more effectively treat solid cancer.

The polypeptide of the present invention may further include a TGF-βR2 exodomain, an IL18R transmembrane domain, and an IL18R endodomain, in addition to a chimeric antigen receptor. In particular, it is preferred that an IL18R transmembrane domain be included between the TGF-βR2 exodomain and the IL18R endodomain (see FIG. 4).

In the present invention, it is preferred that the TGF-βR2 exodomain be linked to a chimeric antigen receptor by a self-cleaving peptide. Therefore, when the polypeptide according to the present invention is expressed in T cells, the polypeptide comprising a TGF-βR2 exodomain, an IL18R transmembrane domain, and an IL18R endodomain is separated from a chimeric antigen receptor (CAR). The TGF-βR2 exodomain of the separated polypeptide is exposed to the outside of T cells, the IL18R transmembrane domain is located in the cell membrane of T cells, and the IL18R endodomain is located in the cytoplasm of T cells.

Accordingly, when TGF-β, which is an immunosuppressive cytokine, is present outside of T cells, TGF-β binds to the TGF-βR2 exodomain, thereby activating the IL18R endodomain. That is, the inhibitory signal of TGF-β (i.e., an immunosuppressive cytokine in a hostile tumor microenvironment) is converted into an activation signal of the cytokine IL-18 in CAR-T cells. Therefore, a polypeptide, which includes the TGF-βR2 exodomain, IL18R transmembrane domain, and IL18R endodomain, becomes a chimeric switch receptor.

IL-18, which is a cytokine, inhibits expression of immunosuppressive materials in T cells and differentiation of regulatory T cells, and enhances immune responses against cancer cells. When the effects of the CAR-T cell therapeutic by IL-18 in advanced solid tumors were examined, it was confirmed that FoxO1 expression in CD4⁺ T cells was decreased by IL-18, and additionally that T-bet expression was increased by IL-18 in CD8⁺ T cells.

Ultimately, IL-18 shows an excellent anticancer effect in advanced solid cancer through the T-bethigh FoxO1low CAR-T, persistent CAR-T cells. In particular, in the present invention, a chimeric switch receptor, which includes a TGF-βR2 exodomain (amino acid positions of 23 to 166; SEQ ID NO: 19), which is a receptor for binding to TGF-β (i.e., an immunosuppressive cytokine around solid cancer); and a transmembrane domain and an endodomain of IL-18R (amino acid positions of 323 to 541; SEQ ID NO: 20), which is a cytokine receptor, was prepared to be expressed in CAR-T cells, thereby changing the immunosuppressive signal into an activation signal in CAR-T cells by making a reverse use of the hostile tumor microenvironment.

As described above, even a polypeptide, which additionally includes a TGF-βR2 exodomain, an IL18R transmembrane domain, and an IL18R endodomain in the chimeric antigen receptor, may further include the cytokine described above (e.g., IL-21).

That is, the cytokine described above (e.g., IL-21) may be linked by a self-cleaving peptide to the IL18R endodomain of the polypeptide, which additionally includes a TGF-βR2 exodomain, an IL18R transmembrane domain, and an IL18R cytoplasmic endodomain in the chimeric antigen receptor.

Accordingly, when the polypeptide according to the present invention is expressed in T cells, the polypeptide which includes the TGF-βR2 exodomain, IL18R transmembrane domain, and IL18R endodomain can be separated from the chimeric antigen receptor (CAR), and additionally, the cytokine can also be independently separated. Therefore, the separated cytokine can be released to the outside of T cells, the TGF-βR2 exodomain is exposed to the outside of the T cells, the IL18R transmembrane domain is located in the cell membrane of T cells, and the IL18R endodomain is located in the cytoplasm of T cells.

The polypeptide according to the present invention may be, for example, a polypeptide represented by any one of SEQ ID NOS: 23 to 30 and 34 to 37.

In addition, the present invention relates to a CAR-T cell in which the CAR-containing polypeptide is expressed as described above.

In addition, the present invention relates to a chimeric antigen receptor (CAR) expression vector, which includes an antigen binding domain; a hinge region; a transmembrane domain; a costimulatory domain; and a cytoplasmic signaling domain. In particular, the CAR expression vector includes a nucleic acid encoding a CAR, in which the nucleic acid encoding a CAR comprises a nucleic acid encoding a 4-1BB domain as the costimulatory domain and a nucleic acid encoding a CD3ζ domain as the signaling domain, and further comprises a nucleic acid encoding IL-7Rα or a part thereof or a nucleic acid encoding IL-2Rβ or a part thereof between the nucleic acid encoding the 4-1BB domain and the nucleic acid encoding the CD3ζ domain. In particular, a part of IL-7Rα may be, for example, a sequence of SEQ ID NO: 15, and a part of IL-2Rβ may be, for example, a sequence of SEQ ID NO: 17.

In the present invention, the nucleic acid encoding the CD3ζ domain may be a nucleic acid encoding a CD3ζ domain, in which in a third ITAM region among the three ITAMs present within the CD3ζ domain, a first motif, YxxL, and a second motif, YxxLHM, are substituted. Preferably, the nucleic acid encoding the CD3ζ domain may be a nucleic acid encoding a CD3ζ domain, in which in a third ITAM region, a first motif, YxxL, is substituted with YxxQ; and a second motif region, YxxLHM, is substituted with YxYVTM; or one in which a first motif, YxxL, is substituted with YyyL, and a second motif region, YxxL, is substituted with YxxQ (in which x and y each represent any amino acid).

In addition, the CAR expression vector of the present invention may further include a nucleic acid encoding cytokine IL-21. In particular, the nucleic acid encoding cytokine IL-21 is linked to a nucleic acid encoding a CAR through a nucleic acid encoding a self-cleaving peptide.

In addition, the CAR expression vector of the present invention may further include a nucleic acid encoding a TGF-βR2 exodomain, a nucleic acid encoding an IL18R transmembrane domain, and a nucleic acid encoding an IL18R endodomain. In particular, the nucleic acid encoding the TGF-βR2 exodomain is linked to a nucleic acid encoding a CAR through a nucleic acid encoding a self-cleaving peptide.

As described above, the CAR expression vector, which includes a nucleic acid encoding a TGF-βR2 exodomain, a nucleic acid encoding an IL18R transmembrane domain, and a nucleic acid encoding an IL18R endodomain, may further include a nucleic acid encoding cytokine IL-21. In particular, the nucleic acid encoding cytokine IL-21 is linked to a nucleic acid encoding an IL18R endodomain through a nucleic acid encoding a self-cleaving peptide.

The present invention relates to a CAR-T cell prepared by introducing the CAR expression vector described above.

In addition, the present invention relates to an anticancer agent containing the CAR-T cell described above. The anticancer agent of the present invention containing the CAR-T cell may further contain a pharmaceutically acceptable additive as necessary.

In the CAR-containing polypeptides according to the present invention, CAR-T cells for various carcinomas may be prepared by changing the cancer antigen target of the antigen binding domain as necessary. For example, the antigen binding domain may be prepared as those which bind to IL13Rα2, an antigen associated with an angiogenesis activity (anti-angiogenesis), and antigens such as EGFRvIII, EphA2, αVβ3, Mesothelin, and glypican1. That is, if any ligand or antibody targeting IL-13Ra2, anti-angiogenesis, EGFRvIII, EphA2, aVβ3, glypican1, and mesothelin (see SEQ ID NOS: 1 to 7 and 33) is introduced, these CAR-T cells may be used as an anticancer agent for these targets. Therefore, it is possible to prepare CAR-T cells having an anti-cancer effect against a specific carcinoma.

In the case of EGFRvIII (i.e., a major tumor antigen expressed in glioblastoma, lung cancer, etc.), in order to reduce side effects of CAR-T cells that appear through a non-specific binding, a CAR expression rate and persistence in CAR-T cells were optimized while simultaneously changing the target sequence so as to minimize the binding affinity with EGFR wild-type while maintaining the specificity for EGFRvIII.

While changing positions 52 to 57 of SEQ ID NO: 3 from STGGYN to DPENDE (a CDR2 part of a heavy chain), position 101 of SEQ ID NO: 3 from S to G (a CDR3 part of a heavy chain), and position 229 of SEQ ID NO: 3 from V to G (a CDR3 part of a light chain) in the target sequence (SEQ ID NO: 33), a CAR expression rate and persistence in CAR-T cells were improved using CD8 signal sequence (SEQ ID NOS: 34 and 35).

In order to increase a CAR expression rate and persistence in CAR-T cells targeting aVβ3, a Gaussia princeps luciferase signal sequence or CD8 signal sequence was used (SEQ ID NOS: 36 and 37).

### Advantageous Effects

The CAR-T cells disclosed in the present invention have an excellent expression rate and persistence; therefore, these cells show the effects of persistence and anti-tumor effects in the human body, thereby having an improved therapeutic effect against solid cancer.

### BRIEF DESCRIPTION OF DRAWINGS

FIG. 1 shows a diagram illustrating a process of effectively treating solid cancer by a CAR-containing polypeptide according to the present invention.

FIG. 2 shows a diagram and a table illustrating an antigen binding domain which can be used for a CAR-containing polypeptide platform according to the present invention.

FIG. 3 shows diagrams illustrating the introduction of a cytokine signaling domain into CAR-T cells so as to control CAR tonic signaling in the CAR-containing polypeptide according to the present invention.

FIG. 4 shows diagrams illustrating the introduction of a chimeric switch receptor so as to convert an immune suppressive signal in a hostile tumor microenvironment into an activation signal in CAR-T cells.

FIG. 5 shows diagrams illustrating the release of cytokine IL-21 to the outside of CAR-T cells so as to aid the activation of innate immune-related cells and to increase the content of less-differentiated memory CAR-T cells.

FIG. 6 shows diagrams illustrating the structures of CAR-containing polypeptides according to the present invention.

FIG. 7 shows diagrams illustrating the self-cleaving peptides used in the CAR-containing polypeptides according to the present invention.

FIG. 8 shows a graph and tables illustrating the growth rate and viability of CAR-T cells, which are transformed with the CAR of the present invention.

FIG. 9 shows graphs and a table illustrating the analysis results of the CAR expression rate of CAR-T cells, which were transformed with the CAR of the present invention, by flow cytometry analysis.

FIG. 10 shows a graph and a table illustrating the analysis results of the chimeric switch receptor expression rate of TGF-βR2 exodomain and transmembrane domain and an endodomain of IL18R of CAR-T cells, which were transformed with the CAR of the present invention, by flow cytometry analysis.

FIG. 11 shows the results of Day 10 phenotypes of CAR-T cells, which were transformed with the CAR of the present invention, by flow cytometry analysis.

FIG. 12 shows a graph, which illustrates the cytotoxicity of CAR-T cells, which were transformed with the CAR of the present invention, confirmed through LDH assay after 24 hours of co-culture with U87 cells (human brain cancer cell line) and 293FT target cells (which were used as a control group for normal cells); and a table, which illustrates the purity and viability of CAR-T cells used in a CAR-T cytotoxicity test.

FIG. 13 shows graphs illustrating the analysis results of spontaneous toxicity of CAR-T cells, which were transformed with the CAR of the present invention, after 24 hours or 96 hours.

FIGS. 14 to 16 each show graphs and a table illustrating the analysis results of changes in a CAR expression rate of CAR-T cells, which were transformed with the CAR of the present invention, after 24 hours (FIG. 14), 48 hours (FIG. 15), and 96 hours (FIG. 16) of co-culture of the CAR-T cells with U87 cells (human brain cancer cell line) and 293FT target cells (which were used as a control group for normal cells), by flow cytometry analysis.

FIG. 17 shows graphs and a table illustrating the analysis results of changes in the chimeric switch receptor expression rate of TGF-βR2 exodomain and transmembrane domain and an endodomain of IL-18R of CAR-T cells, which were transformed with the CAR of the present invention, after 24 hours of co-culture of the CAR-T cells, which were transformed with the CAR of the present invention, with U87 cells (human brain cancer cell line) and 293FT target cells (which were used as a control group for normal cells), by flow cytometry analysis.

FIG. 18 shows graphs illustrating the comparison results of IFN-γ cytokine production after 24 hours and 48 hours of co-culture of CAR-T cells, which were transformed with the CAR of the present invention, with U87 cells (human brain cancer cell line) and 293FT target cells (which were used as a control group for normal cells).

FIG. 19 shows graphs illustrating the comparison results of IL-21 cytokine production after 24 hours and 48 hours of co-culture of CAR-T cells, which were transformed with the CAR of the present invention, with U87 cells (human brain cancer cell line) and 293FT target cells (which were used as a control group for normal cells).

FIG. 20 shows a graph and tables illustrating the growth rate and viability of CAR-T cells, which were transformed with a chimeric antigen receptor (CAR), when the cancer antigen is EGFRvIII, in which the CAR expression rate and persistence in CAR-T cells were optimized, while simultaneously changing the target sequence so as to minimize the binding affinity for EGFR wild-type, where the specificity for EGFRvIII is maintained so as to reduce the side effects of CAR-T cells that appear through non-specific binding (YYB105, #13, #14) (see SEQ ID NOS: 39, 34, 35); and a graph and tables illustrating the growth rate and viability of CAR-T cells, which were transformed with a chimeric antigen receptor (CAR), when the cancer antigen is aVβ3, in which the CAR expression rate in CAR-T cells was optimized (YYB107, #15, #16) (see SEQ ID NOS: 38, 36, 37).

FIG. 21 shows a graph illustrating the CAR expression rate in CAR-T cells, which were transformed with a chimeric antigen receptor (CAR), when the cancer antigen is EGFRvIII, in which the CAR expression rate in CAR-T cells was optimized, while simultaneously changing the target sequence so as to minimize the binding affinity for EGFR wild-type, where the specificity for EGFRvIII is maintained so as to reduce the side effects of CAR-T cells that appear through non-specific binding (YYB105, #13, #14) (see SEQ ID NOS: 39, 34, 35); and a graph illustrating the CAR expression rate in CAR-T cells, which were transformed with a chimeric antigen receptor (CAR), when the cancer antigen is aVβ3, in which the CAR expression rate in CAR-T cells was optimized (YYB107, #15, #16) (see SEQ ID NOS: 38, 36, 37).

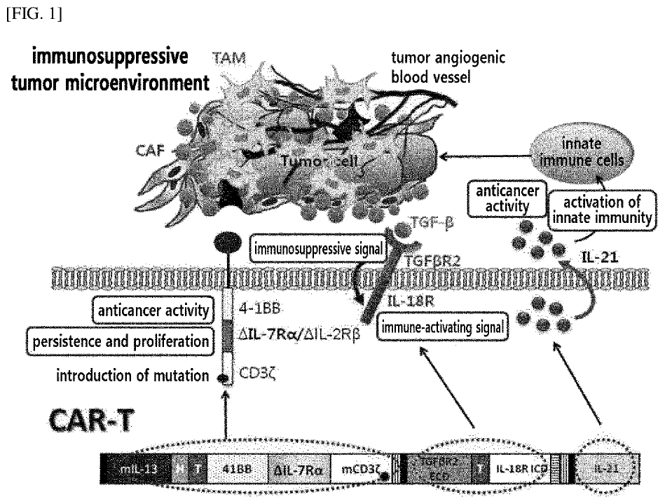

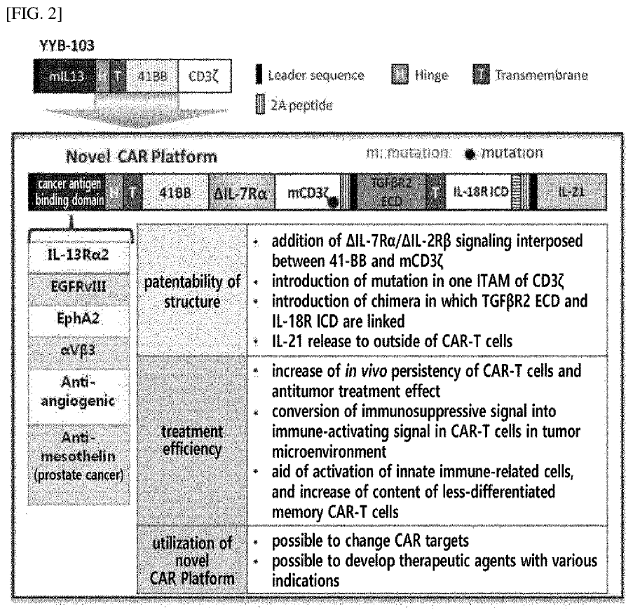

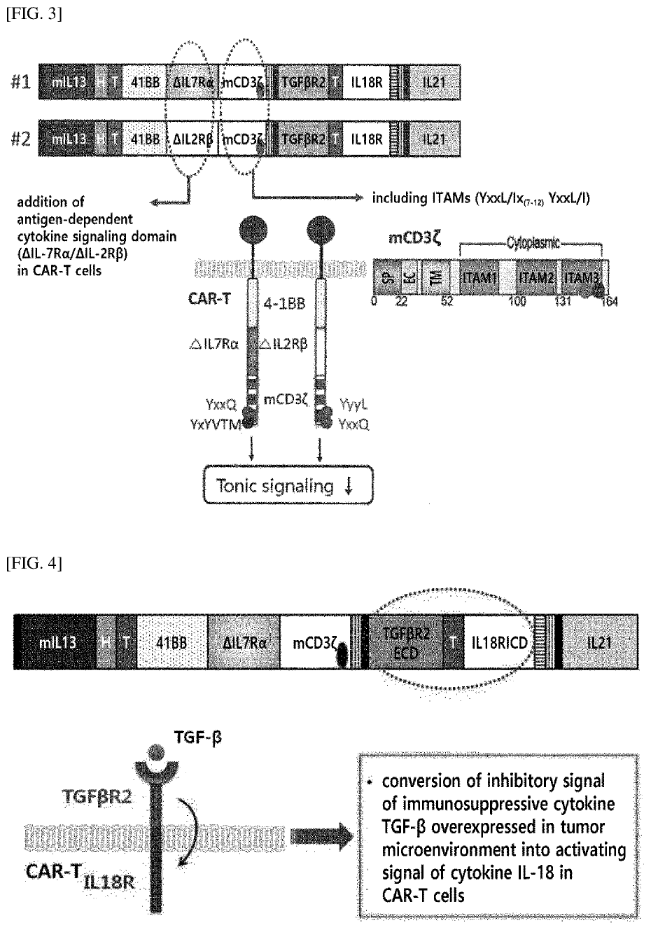

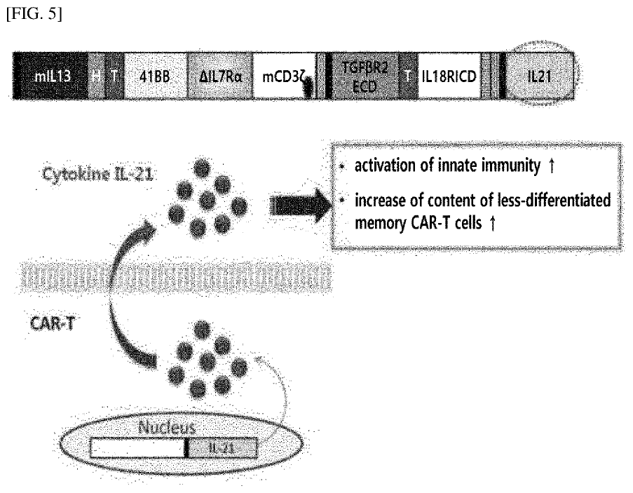

### BEST MODE FOR CARRYING OUT THE INVENTION

Hereinafter, the present invention will be described in detail through examples. However, it should be noted that the following Examples are only for illustrating the present invention, and the spirit and technical scope of the present invention are not limited in any sense. Further, it should be understood that the present invention is not limited to the precise arrangement and means of the embodiments shown in the drawings, which are cited as reference.

### Example 1: Preparation of Novel Chimeric Antigen Receptor (CAR) Platform for Effective Treatment by Specifically Binding to IL13Rα2 Overexpressed in Solid Cancer Cells

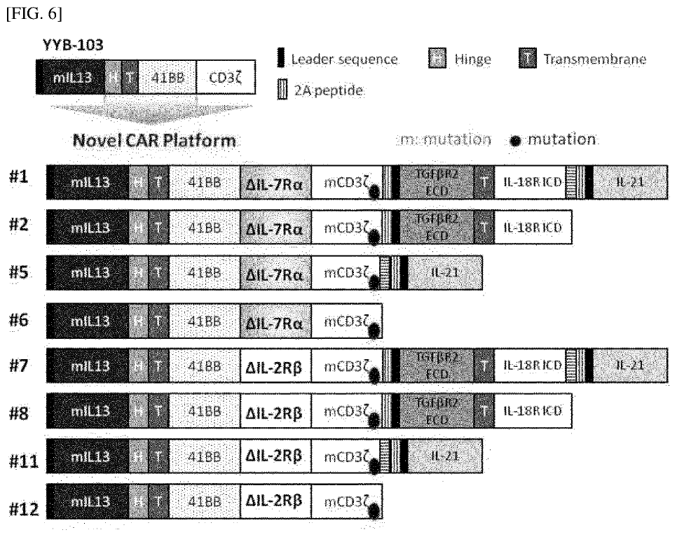

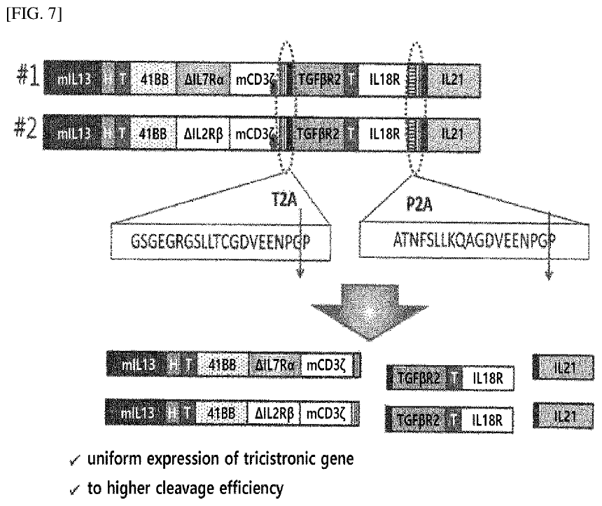

With regard to human IL13 (P35225.1), human CD3ζ (P20963-1), human CD8A (P01732), human CD28 (P10747), human CD3ζ (P20963), human 41BB (Q07011), IL7RA (P16871), IL2RB (P14784), TGFR2 (P37173), IL18R (Q13478), IL21 (Q9HBE4), IL2 (P60568), T2A, P2A, and human kappa light chain signal sequence (HuVHCAMP), CAR-containing polypeptides were prepared using a chimeric antigen-containing polypeptide (see SEQ ID NOS: 23 to 30 and 34 to 37; and #1, #2, #5, #6, #7, #8, #11, and #12 of FIG. 6) expression vector consisting of codon-optimized synthetic DNA, using scientific literature and publicly available databases (see FIGS. 6 and 7).

Specifically, T cells, in which a CAR-containing polypeptide is expressed, can be prepared by preparing a vector that expresses a CAR-containing polypeptide via conjugation of synthetic DNA to an MFG retrovirus expression vector digested with XhoI/NotI, followed by transduction of the vector into the T cells.

The completed structure of polypeptide #1 of FIG. 6 includes a Kozak consensus ribosome-binding sequence, a human kappa light chain signal sequence (HuVHCAMP), human IL13.E11K.R64D.S67D.R107K mature protein sequence (see YYB-103 of PCT International Publication No. WO 2017/023138), three glycines (GGG) which are introduced between an antigen binding domain and a hinge region so as to increase the expression of a chimeric antigen receptor by increasing the solubility of a CAR protein (see YYB-103 of PCT International Publication No. WO 2017/023138), a hinge region of human CD8a, a human CD8 transmembrane domain, a costimulatory signal domain of cytoplasmic 4-1BB, a cleaved cytoplasmic domain of cytokine IL-7RA (amino acid positions of 265 to 328; SEQ ID NO: 15), a mutant CD3ζ (positions 91 to 113 of SEQ ID NO: 18 are mutated to a sequence of SEQ ID NO: 31), T2A, a CD8A signal sequence (leader sequence), a chimeric switch receptor which comprises TGF-βR2 exodomain (amino acid positions of 23 to 166; SEQ ID NO: 19) and a transmembrane domain and an endodomain of IL-18R (i.e., a cytokine receptor) (amino acid positions of 323 to 541; SEQ ID NO: 20), P2A, a human IL2 signal sequence (leader sequence), an IL-21 (amino acid positions of 23 to 155; SEQ ID NO: 21) sequence, and an XhoI/NotI cleavage site (see CAR-containing polypeptide #1 of FIG. 6; and FIG. 7).

The completed structure of CAR-containing polypeptide #2 of FIG. 6 includes a Kozak consensus ribosome-binding sequence, a human kappa light chain signal sequence (HuVHCAMP), human IL13.E11K.R64D.S67D.R107K mature protein sequence, three glycines (GGG) which are introduced between an antigen binding domain and a hinge region so as to increase the expression of a chimeric antigen receptor by increasing the solubility of a CAR protein, a hinge region of human CD8α, a human CD8 transmembrane domain, a costimulatory signal domain of cytoplasmic 4-1BB, a cleaved cytoplasmic domain of a cytokine IL-7RA (amino acid positions of 265 to 328; SEQ ID NO: 15), a mutant CD3ζ (positions 91 to 113 of SEQ ID NO: 18 are mutated to a sequence of SEQ ID NO: 31), T2A, a CD8A signal sequence (leader sequence), a chimeric switch receptor which comprises TGF-βR2 exodomain (amino acid positions of 23 to 166; SEQ ID NO: 19) and a transmembrane domain and an endodomain of IL-18R (i.e., a cytokine receptor) (amino acid positions of 323 to 541; SEQ ID NO: 20), and an XhoI/NotI cleavage site (see CAR-containing polypeptide #2 of FIG. 6; and FIG. 7).

The completed structure of CAR-containing polypeptide #5 of FIG. 6 includes a Kozak consensus ribosome-binding sequence, a human kappa light chain signal sequence (HuVHCAMP), human IL13.E11K.R64D.S67D.R107K mature protein sequence, three glycines (GGG) which are introduced between an antigen binding domain and a hinge region so as to increase the expression of a chimeric antigen receptor by increasing the solubility of a CAR protein, a hinge region of human CD8a, a human CD8 transmembrane domain, a costimulatory signal domain of cytoplasmic 4-1BB, a cleaved cytoplasmic domain of a cytokine IL-7RA (amino acid positions of 265 to 328; SEQ ID NO: 15), a mutant CD3ζ (positions 91 to 113 of SEQ ID NO: 18 are mutated to a sequence of SEQ ID NO: 31), T2A, a human IL2 signal sequence, an IL-21 (amino acid positions of 23 to 155; SEQ ID NO: 21) sequence, and an XhoI/NotI cleavage site (see CAR-containing polypeptide #5 of FIG. 6; and FIG. 7).

The completed structure of CAR-containing polypeptide #6 of FIG. 6 includes a Kozak consensus ribosome-binding sequence, a human kappa light chain signal sequence (HuVHCAMP), human IL13.E11K.R64D.S67D.R107K mature protein sequence, three glycines (GGG) which are introduced between an antigen binding domain and a hinge region so as to increase the expression of a chimeric antigen receptor by increasing the solubility of a CAR protein, a hinge region of human CD8α, a human CD8 transmembrane domain, a costimulatory signal domain of cytoplasmic 4-1BB, a cytoplasmic domain of a cytokine IL-7RA (amino acid positions of 265 to 328; SEQ ID NO: 15), a mutant CD3ζ (positions 91 to 113 of SEQ ID NO: 18 are mutated to a sequence of SEQ ID NO: 31), and an XhoI/NotI cleavage site (see CAR-containing polypeptide #6 of FIG. 6; and FIG. 7).

The completed structure of polypeptide CAR-containing #7 of FIG. 6 includes a Kozak consensus ribosome-binding sequence, a human kappa light chain signal sequence (HuVHCAMP), human IL13.E11K.R64D.S67D.R107K mature protein sequence, three glycines (GGG) which are introduced between an antigen binding domain and a hinge region so as to increase the expression of a chimeric antigen receptor by increasing the solubility of a CAR protein, a hinge region of human CD8α, a human CD8 transmembrane domain, a costimulatory signal domain of cytoplasmic 4-1BB, a cleaved cytoplasmic domain of a cytokine IL2Rβ (amino acid positions of 266 to 369; SEQ ID NO: 17), a mutant CD3ζ (positions 91 to 113 of SEQ ID NO: 18 are mutated to a sequence of SEQ ID NO: 32), T2A, a CD8A signal sequence, a chimeric switch receptor which comprises a TGF-βR2 exodomain (amino acid positions of 23 to 166; SEQ ID NO: 19) and a transmembrane domain and an endodomain of IL-18R (i.e., a cytokine receptor) (amino acid positions of 323 to 541; SEQ ID NO: 20), P2A, a human IL2 signal sequence (leader sequence), an IL-21 (amino acid positions of 23 to 155; SEQ ID NO: 21) sequence, and an XhoI/NotI cleavage site (see #7 of FIG. 6; and FIG. 7).

The completed structure of CAR-containing polypeptide #8 of FIG. 6 includes a Kozak consensus ribosome-binding sequence, a human kappa light chain signal sequence (HuVHCAMP), human IL13.E11K.R64D.S67D.R107K mature protein sequence, three glycines (GGG) which are introduced between an antigen binding domain and a hinge region so as to increase the expression of a chimeric antigen receptor by increasing the solubility of a CAR protein, a hinge region of human CD8α, a human CD8 transmembrane domain, a costimulatory signal domain of cytoplasmic 4-1BB, a cleaved cytoplasmic domain of a cytokine IL2Rβ (amino acid positions of 266 to 369; SEQ ID NO: 19), a mutant CD3ζ (positions 91 to 113 of SEQ ID NO: 18 are mutated to a sequence of SEQ ID NO: 32), T2A, a CD8A signal sequence, a chimeric switch receptor which comprises a TGF-βR2 exodomain (amino acid positions of 23 to 166; SEQ ID NO: 19) and a transmembrane domain and an endodomain of IL-18R (i.e., a cytokine receptor) (amino acid positions of 323 to 541; SEQ ID NO: 20), and an XhoI/NotI cleavage site (see CAR-containing polypeptide #8 of FIG. 6; and FIG. 7).

The completed structure of CAR-containing polypeptide #11 of FIG. 6 includes a Kozak consensus ribosome-binding sequence, a human kappa light chain signal sequence (HuVHCAMP), human IL13.E11K.R64D.S67D.R107K mature protein sequence, three glycines (GGG) which are introduced between an antigen binding domain and a hinge region so as to increase the expression of a chimeric antigen receptor by increasing the solubility of a CAR protein, a hinge region of human CD8α, a human CD8 transmembrane domain, a costimulatory signal domain of cytoplasmic 4-1BB, a cleaved cytoplasmic domain of a cytokine IL2Rβ (amino acid positions of 266 to 369; SEQ ID NO: 17), a mutant CD3ζ (positions 91 to 113 of SEQ ID NO: 18 are mutated to a sequence of SEQ ID NO: 32), T2A, a human IL2 signal sequence, an IL-21 (amino acid positions of 23 to 155; SEQ ID NO: 21) sequence, and an XhoI/NotI cleavage site (see CAR-containing polypeptide #11 of FIG. 6; and FIG. 7).

The completed structure of polypeptide CAR-containing #12 of FIG. 6 includes a Kozak consensus ribosome-binding sequence, a human kappa light chain signal sequence (HuVHCAMP), human IL13.E11K.R64D.S67D.R107K mature protein sequence, three glycines (GGG) which are introduced between an antigen binding domain and a hinge region so as to increase the expression of a chimeric antigen receptor by increasing the solubility of a CAR protein, a human hinge region of CD8α, a human CD8 transmembrane domain, a costimulatory signal domain of cytoplasmic 4-1BB, a cleaved cytoplasmic domain of a cytokine IL2Rβ (amino acid positions of 266 to 369; SEQ ID NO: 17), a mutant CD3ζ (positions 91 to 113 of SEQ ID NO: 18 are mutated to a sequence of SEQ ID NO: 32), and an XhoI/NotI cleavage site (see #12 of FIG. 6; and FIG. 7).

The entire sequences of CAR-containing polypeptides #1, #2, #5, #6, #7, #8, #11, and #12 are shown in SEQ ID NOS: 23 to 30.

The finally prepared CAR gene fragments were conjugated to the MFG retrovirus expression vector digested with XhoI/NotI (Emtage P C et al., Clin Cancer Res, 2008, 14:8112-8122). In this Example, in order to compare the activity of chimeric antigen receptors, YYB103 (SEQ ID NO: 22) was further prepared.

### Example 2: Preparation of CAR-T Cells Transformed with Novel Chimeric Antigen Receptors

High-titer CAR-expressing PG13 clones were prepared such that Phoenix-Ampho and Phoenix-Eco cells were transiently transfected with the retroviral expression vector prepared in Example 1, and then, cell-free vector stocks were prepared from the transfected Phoenix-Ampho and Phoenix-Eco cells by transfecting PG13 cells.

For high-titer monoclones, PG13/#1, PG13/#2, PG13/#5, PG13/#6, PG13/#7, PG13/#8, PG13/#11, or PG13/#12 cells were stained using an anti-IL-13 monoclonal antibody (BD Pharmingen), and these single clones were isolated using a flow cytometer. The PG13/#1, PG13/#2, PG13/#5, PG13/#6, PG13/#7, PG13/#8, PG13/#11, or PG13/#12 clones were prepared by the second subcloning according to the limiting dilution method. These subclones stably showed high CAR expression and were selected for the efficient transduction ability in the peripheral blood.

The transduction degree of PG13/#1, PG13/#2, PG13/#5, PG13/#6, PG13/#7, PG13/#8, PG13/#11, or PG13/#12 cells, which were transduced using an anti-IL-13 monoclonal antibody (BD Pharmingen), was analyzed using a flow cytometer. The supernatants of the transduced PG13/#1, PG13/#2, PG13/#5, PG13/#6, PG13/#7, PG13/#8, PG13/#11, or PG13/#12 cells contained retrovirus, and the supernatants were collected for genetic modification of T cells.

The peripheral blood mononuclear cells (PBMCs) were separated using centrifugation by adding the whole blood obtained from a healthy human donor into Ficoll Paque (GE Healthcare). The separated PBMCs were cultured by adding an anti-CD3 monoclonal antibody (eBioscience) at a concentration of 100 ng/mL under the condition of human IL-2 (Novartis) at a concentration of 100 IU/mL to activate the T cell fraction (BL Levine, Cancer Gene Therapy, 2015, 22:79-84). Two to three days after the cultivation, most of the cells were T cells and included natural killer cells at a percentage of 0% to 2%. Two to three days after the activation step, the T cells were subjected to transduction two times over two days using the retroviral supernatant and washed, and then proliferated for four to seven days in a flask. The cells were cultured in a stirring platform device (a WAVE bio-reactor system) for 12 to 28 days. IL-2 was maintained at a concentration of 100 IU/mL. The T cells modified as such were used for an analysis experiment.

### Experimental Example 1: Checking of Growth Rate and Viability of CAR-T Cells Transformed with Novel Chimeric Antigen Receptors

Experimental Results

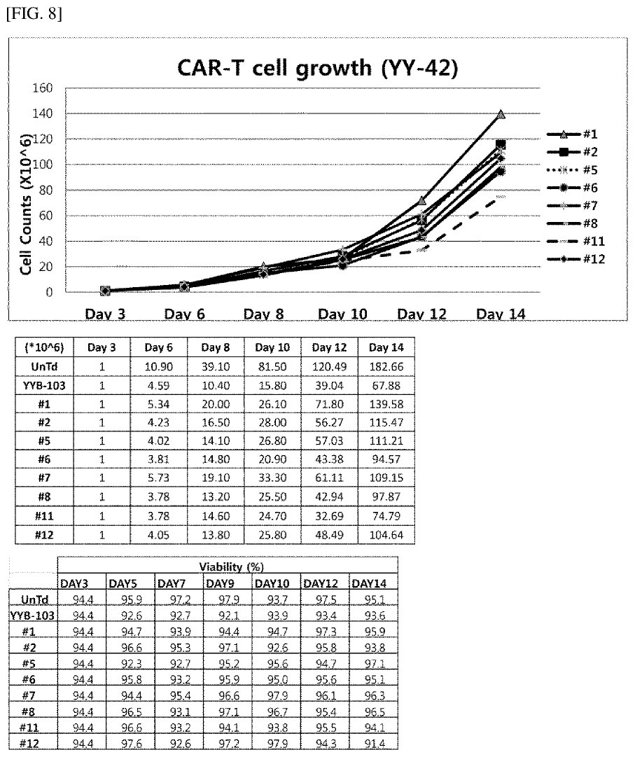

For the T cells prepared in Example 2, the number of cells was counted to confirm the growth rate and viability of CAR-T, and the results are shown in FIG. 8.

The number of cells and growth rate of all of the groups (#1, #2, #5, #6, #7, #8, #11, or #12) were shown to be higher than those of the control group (i.e., YYB103) depending on the day of the week, the cells were shown to grow rapidly from Day 12 of culture, and the viability was also shown to be 90% or higher (see FIG. 8).

### Experimental Example 2: Checking of CAR Expression Rate on Surface of CAR-T Cells Transformed with Novel Chimeric Antigen Receptors

Experimental Method (Flow Cytometric Analysis)

For flow cytometry (>30,000 events), a BD LSRII device (Becton Dickinson) and BD FACSDiva software (Becton Dickinson) were used. Specifically, the cells were washed once with PBS containing 2% bovine serum albumin before adding a PE-conjugated anti-human IL-13 monoclonal antibody (BD Pharmingen) thereto. After washing, the cells were reacted with each antibody at 4° C. for 30 minutes in a state where light was blocked, and washed once, and thereafter, the expression rate of CAR on the surface of transduced T cells was checked.

Experimental Results

In order to confirm whether the 8 kinds of IL13Rα2-specific, CAR-containing polypeptides prepared in Example 2 (#1, #2, #5, #6, #7, #8, #11, or #12) were expressed on the T cell surface, T cell culture was performed for 28 days according to Example 2, and then flow cytometric analysis was performed according to the experimental method.

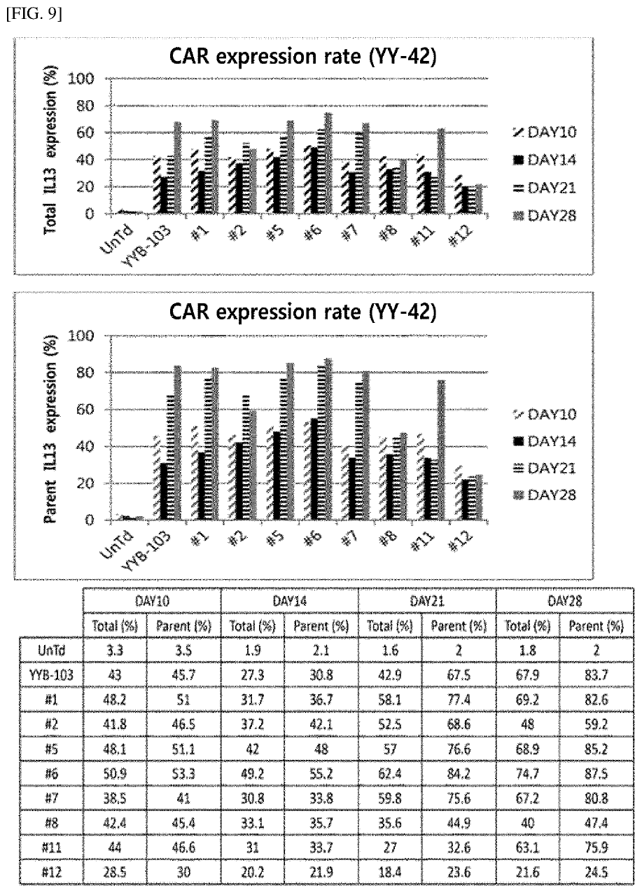

As a result of the analysis, as shown in FIG. 9, the expression rate of the chimeric antigen receptors expressed on the cell surface of live T cells was shown to be in the range of 24.5% to 84.2%, and the expression of IL13Rα2-specific chimeric antigen receptors was stably maintained for 4 weeks, without additional activation or transduction of T cells (see FIG. 9).

### Experimental Example 3: Checking of Chimeric Switch Receptor Expression Rates of TGF-βR2 Exodomain and Transmembrane Domain and an Endodomain of IL-18R on the Cell Surface of CAR-T Cells Transformed with Novel Chimeric Antigen Receptors

Experimental Methods (flow cytometric analysis) For flow cytometry (>30,000 events), a BD LSRII device (Becton Dickinson) and BD FACSDiva software (Becton Dickinson) were used. Specifically, the cells were washed once with PBS containing 2% bovine serum albumin before adding a human TGF-βR2 fluorescein-conjugated antibody (FAB2411F) (BD Pharmingen) thereto. After washing, the cells were reacted with each antibody at 4° C. for 30 minutes in a state where light was blocked and washed once, and thereafter, the chimeric switch receptor expression rates of TGF-βR2 exodomain and transmembrane domain and an endodomain of IL-18R on the cell surface of transduced T cells were checked.

Experimental Results

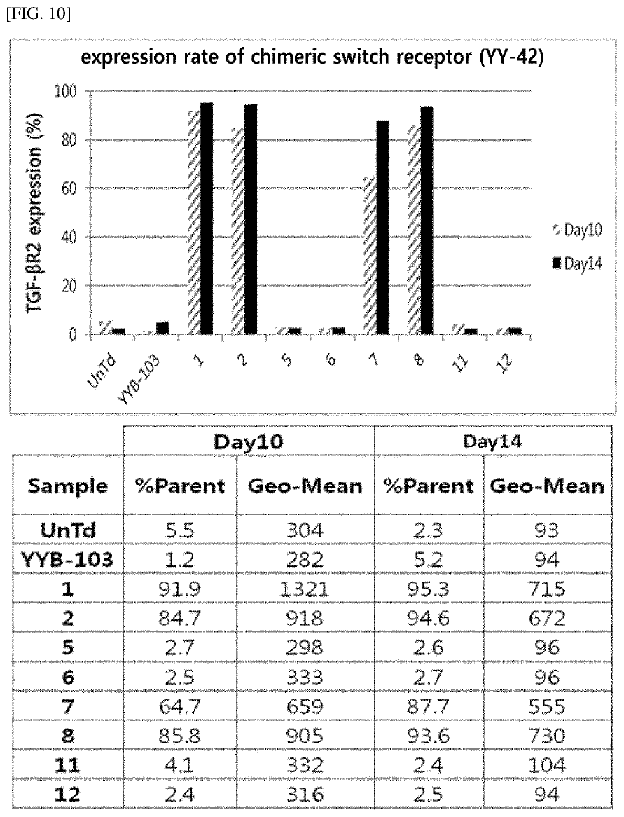

In order to confirm whether the chimeric switch receptors of TGFβR2 exodomain and transmembrane domain and an endodomain of IL-18R were expressed on the cell surface of CAR-T cells prepared in Example 2 (#1, #2, #5, #6, #7, #8, #11, or #12), T cell cultivation was performed for 14 days according to Example 2, and then flow cytometric analysis was performed according to the experimental method. As a result of the analysis, the expression rates of the chimeric switch receptors of TGFβR2 exodomain and transmembrane domain and an endodomain of IL-18R on the cell surface of live T cells were shown to be about 85% (see FIG. 10).

### Experimental Example 4: Checking of Phenotypes on the Cell Surface of CAR-T Cells Transformed with Novel Chimeric Antigen Receptors

Experimental Methods (Flow Cytometric Analysis)

For flow cytometry (>30,000 events), a BD LSRII device (Becton Dickinson) and a BD FACSDiva software (Becton Dickinson) were used. Specifically, the cells were washed once with PBS containing 2% bovine serum albumin before adding an FITC-conjugated CD45RA Ab (HI100) (Biolegend), a PE-conjugated CCR7Ab (G043H7) (Biolegend), and a PE-Cy7-conjugated CD62L Ab (DREG-56) (Biolegend) thereto. After washing, the cells were reacted with each antibody at 4° C. for 30 minutes in a state where light was blocked and washed once, and thereafter, the content of less-differentiated memory CAR-T cells and CCR7⁺CD45RA⁺CD62L⁺ phenotypes on the surface of transduced T cells were checked.

Experimental Results

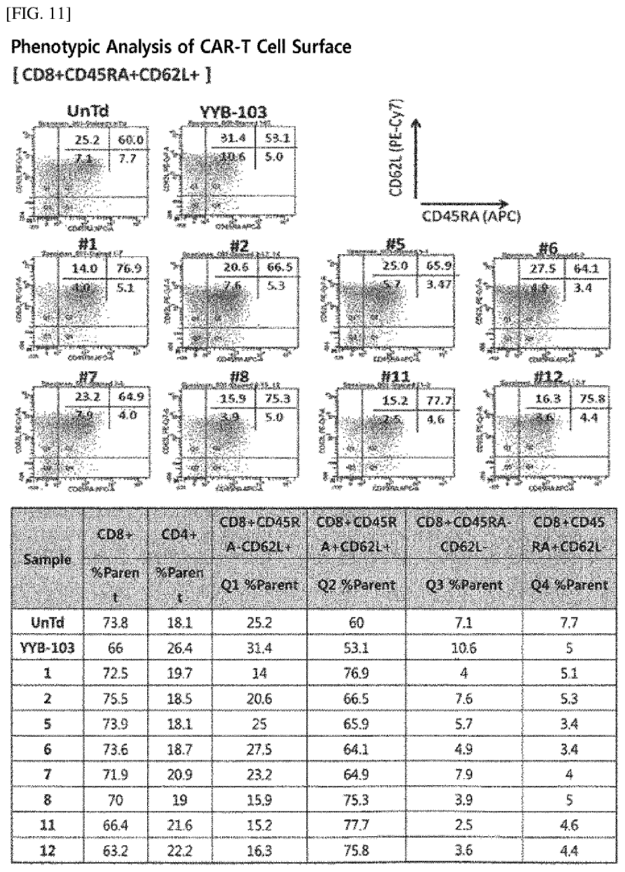

In order to confirm the content of the less-differentiated memory CAR-T cells on the cell surface of CAR-T cells prepared in Example 2 (#1, #2, #5, #6, #7, #8, #11, or #12), T cell cultivation was performed for 10 days according to Example 2, and then flow cytometric analysis was performed according to the experimental method. As a result of the analysis, the content of less-differentiated memory CAR-T cells showing CCR7⁺CD45RA⁺CD62L⁺ phenotypes, which were expressed on live T cells, compared to a non-transduced sample (i.e., a control group) by 5% or higher, and compared to YYB103 (i.e., the transduced sample, CAR-T control group) by 10% or higher, on the surface of all of the groups of CAR-T cells prepared in Example 2 (#1, #2, #5, #6, #7, #8, #11, or #12), was confirmed (see FIG. 11).

### Experimental Example 5: Checking of Cytotoxicity of CAR-T Cells Transformed with Novel Chimeric Antigen Receptors Against Glioma Cell Line in which IL13Rα2 is Overexpressed

Experimental Methods

In order to measure the cytotoxicity of IL13Rα2-specific CAR-T cells prepared in Example 2 (#1, #2, #5, #6, #7, #8, #11, or #12), cytotoxicity assay was performed using an LDH (Promega) kit. Specifically, CAR-T cells (effector cells) were used 10 days after activation of the cells with an anti-CD3 Ab, and the CAR-T cells (having a CAR expression rate of 20% to 40%) were added to a 6-well plate at a ratio 1:2, in which effector:target was 1×10⁶ cells:2×10⁶ cells and reacted at 37° C. for 24 hours. As the used target cells, U87 cells (human brain cancer cell line expressing IL13Rα2) and 293FT cells (the control group for normal cells) were used.

Experimental Results

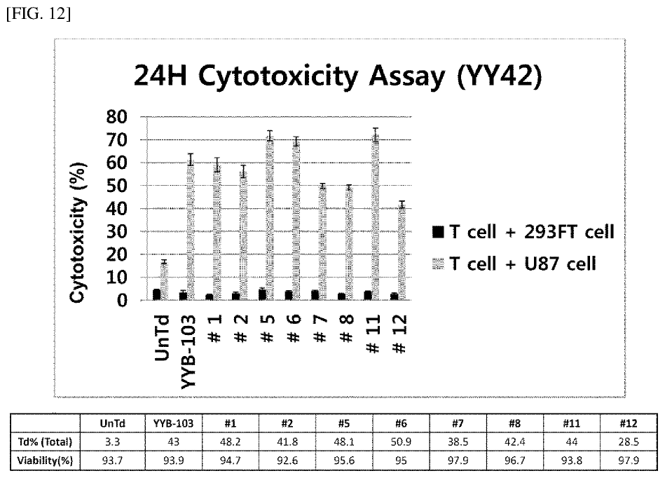

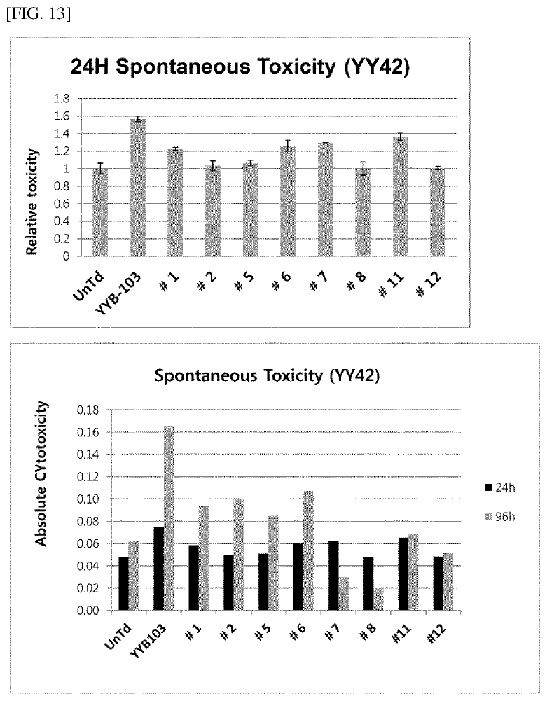

An analysis was performed to examine whether IL13Rα2-specific CAR-T cells prepared in the present invention can effectively kill the target cancer cells (U87 expressing IL13Rα2). The method used was such that where target cancer cells (U87 expressing IL13Rα2) and normal cells (293FT) described above were cultured together with each of the activated CAR-T cells and cytotoxicity was compared and analyzed. As shown in FIG. 12, all of the CAR-T cells prepared according to the present invention showed a higher effect by 50% to 70% of inducing the death of the target cancer cell (U87) compared to non-transduced activated T cells. In particular, CAR-T #5, #6, and #11 of the present invention showed high cytotoxicity. In addition, CAR-T cells (#1, #2, #5, and #6) containing IL-7Rα exhibited higher overall cytotoxicity compared to CAR-T cells (#7, #8, #11, and #12) containing IL-2Rβ.

From the experimental result where 293FT cells, which do not express IL13Rα2, were used as the control group for normal cells, as an object to be compared with the target cells, it was confirmed that the CAR-T cells specific to IL13Rα2 showed very weak cytotoxicity (2% to 4%). This result shows that the chimeric antigen receptors used in this experiment bind specifically to IL13Rα2.

Through this Experimental Example, it was found that IL13Rα2-specific chimeric antigen receptor T cells do not show cytotoxicity to a normal cell (293FT) and significantly kill target cancer cells (U87), which express IL13Rα2.

FIG. 13 shows the results in which the cytotoxicity of the CAR-T cells, which were transformed with the chimeric antigen receptor (CAR) of the present invention, was confirmed through LDH assay after 24 hours or 96 hours without co-culture with target cells. Since the spontaneous toxicity of YYB103 is higher than that of other groups, the 8 groups are expected to show higher cytotoxicity compared to YYB103 in the co-culture with U87 cells, and the CAR-T cells (#1, #2, #5, #6, #7, #8, #11, or #12), which were transformed with the chimeric antigen receptor (CAR) of the present invention, are expected to have a comparative advantage in terms of safety and stability.

### Experimental Example 6: Checking of Changes in CAR Expression Rate when CAR-T Cells, which were Transformed with Novel Chimeric Antigen Receptors, are Co-Cultured with Human Glioma Cell Line in which IL13Rα2 is Overexpressed

Experimental Methods In order to check the changes in CAR expression rate when the CAR-T cells prepared in Example 2 (#1, #2, #5, #6, #7, #8, #11, or #12) are co-cultured with human glioma cell line in which IL13Rα2 is overexpressed, the CAR-T cells (effector cells) having a CAR expression rate of 20% to 40% were used 10 days after activation of the cells with an anti-CD3 Ab. The CAR-T cells were added to a 6-well plate at a ratio 1:2, in which effector:target was 1×10⁶ cells:2×10⁶ cells and reacted at 37° C. for 24, 48, and 96 hours. To the sample, which was reacted for 96 hours, were added 2×10⁶ cells of the target cells after 48 hours, and a BD LSRII device (Becton Dickinson) and BD FACSDiva software (Becton Dickinson) were used for flow cytometry (>30,000 events). Specifically, the cells were washed once with PBS containing 2% bovine serum albumin before adding a PE-conjugated anti-human IL-13 monoclonal antibody (BD Pharmingen) thereto. After washing, the cells were reacted with each antibody at 4° C. for 30 minutes in a state where light was blocked and washed once, and thereafter, the changes in the CAR expression rate were checked.

Experimental Results

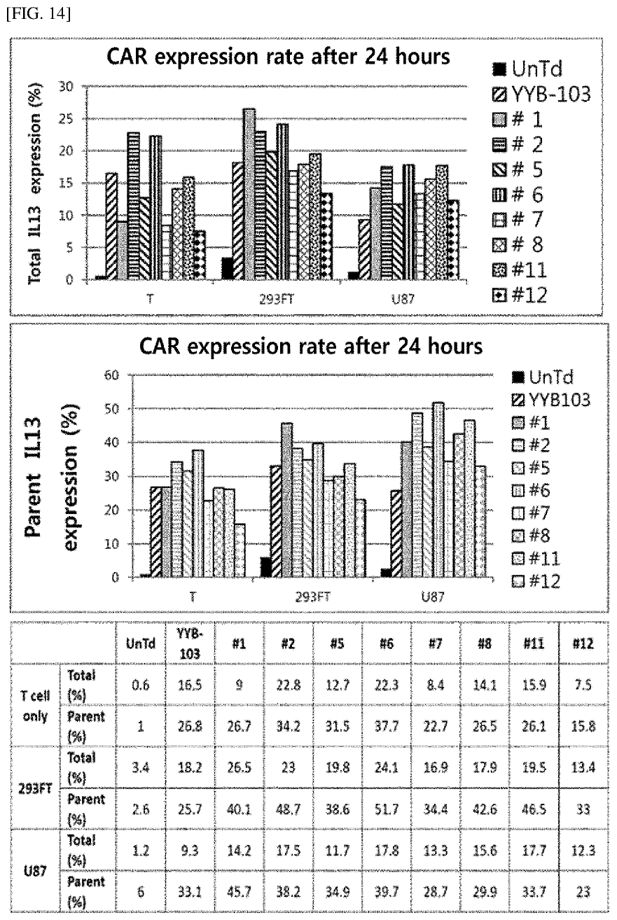

In order to confirm the changes in the CAR expression rate when the 8 kinds of IL13Rα2-specific CAR-T cells prepared in Example 2 (#1, #2, #5, #6, #7, #8, #11, or #12) are co-cultured with human glioma cell line in which IL13Rα2 is overexpressed, the T cells were cultured for 10 days, and then flow cytometric analysis was performed according to the experimental method according to Example 2. As a result of the analysis, as shown in FIG. 14, the CAR-T cells which were co-cultured with U87 (human glioma cell line cells for 24 hours showed a smaller change in CAR expression in all of the 8 groups compared to that of YYB103 (FIG. 14).

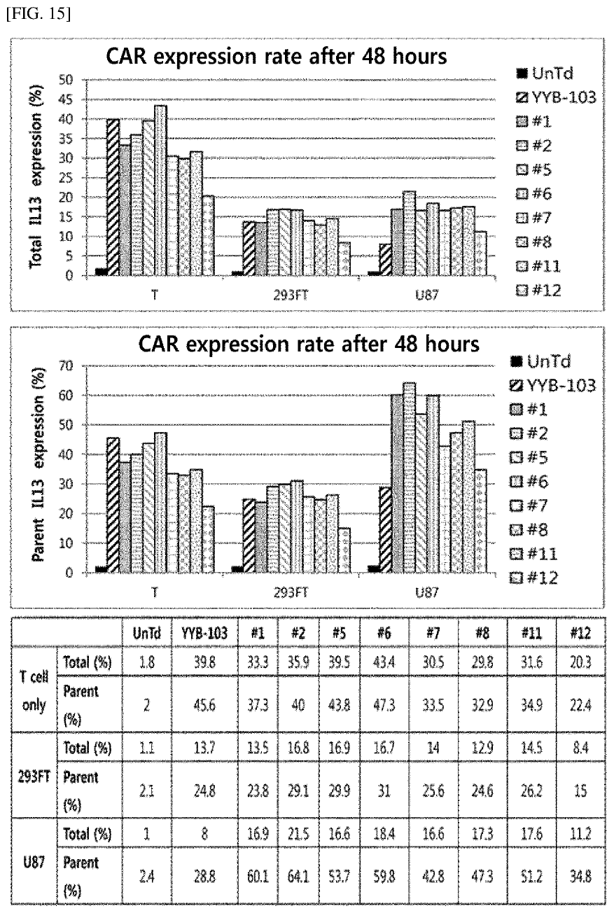

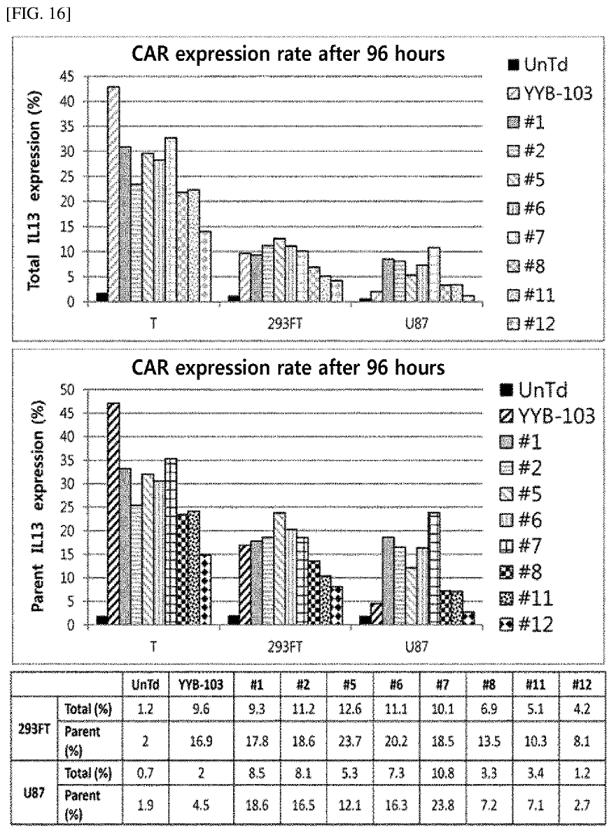

In co-culture with U87 (human glioma cell line) cells for 48 hours, the change in CAR expression was shown to be smaller than that of YYB103 in all of the 8 groups (FIG. 15).

In co-culture with U87 (human glioma cell line) cells for 96 hours, the change in CAR expression was shown to be smaller than that of YYB103 in 5 groups (i.e., #1, #2, #5, #6, and #7) (FIG. 16). The CAR expression of YYB103 after co-culture with U87 (human glioma cell line) cells for 96 hours was shown to be insignificant (FIG. 16).

The changes in CAR expression during the co-culture with human glioma cell line, in which IL13Rα2 is overexpressed, the changes in the CAR expression rate without co-culture with target cells or during the co-culture with 293FT cells (which were used as the control group for normal cells) can confirm that the chimeric antigen receptor (CAR) according to the present invention is expected to show excellent in vivo persistence and anti-tumor effects of CAR-T cells in a hostile tumor microenvironment, while simultaneously reducing toxicity.

### Experimental Example 7: Checking of Changes in Chimeric Switch Receptor Expression Rate of TGFβR2 Exodomain and Transmembrane Domain and an Endodomain of IL-18R when CAR-T Cells which were Transformed with Novel Chimeric Antigen Receptors are Co-Cultured with Human Glioma Cell Line, in which IL13Rα2 is Overexpressed

Experimental Methods

In order to check the changes in the chimeric switch receptor expression rate of TGFβR2 exodomain and transmembrane domain and an endodomain of IL-18R when CAR-T cells prepared in Example 2 (#1, #2, #5, #6, #7, #8, #11, or #12) are co-cultured with glioma cell line, in which IL13Rα2 is overexpressed, the CAR-T cells (effector cells) having a CAR expression rate of 20% to 40% were used 10 days after activation of the cells with an anti-CD3 Ab. The CAR-T cells were added to a 6-well plate at a ratio 1:2, in which effector:target was 1×10⁶ cells:2×10⁶ cells and reacted at 37° C. for 24, 48, and 96 hours. To the sample, which was reacted for 96 hours, were added 2×10⁶ cells of the target cells after 48 hours, and a BD LSRII device (Becton Dickinson) and BD FACSDiva software (Becton Dickinson) were used for flow cytometry (>30,000 events). Specifically, the cells were washed once with PBS containing 2% bovine serum albumin before adding an anti-human TGF-βR2 fluorescein-conjugated antibody (FAB2411F) (BD Pharmingen) thereto. After washing, the cells were reacted with each antibody at 4° C. for 30 minutes in a state where light was blocked and washed once, and thereafter, the changes in the chimeric switch receptor expression rate of TGFβR2 exodomain and transmembrane domain and an endodomain of IL-18R were checked.

Experimental Results

In order to confirm the changes in the chimeric switch receptor expression rate of TGFβR2 exodomain and transmembrane domain and an endodomain of IL-18R when the 8 kinds of IL13Rα2-specific CAR prepared in Example 2 (#1, #2, #5, #6, #7, #8, #11, or #12) are co-cultured with human glioma cell line in which IL13Rα2 is overexpressed, the T cells were cultured for 10 days according to Example 2, and then flow cytometric analysis was performed according to the experimental method.

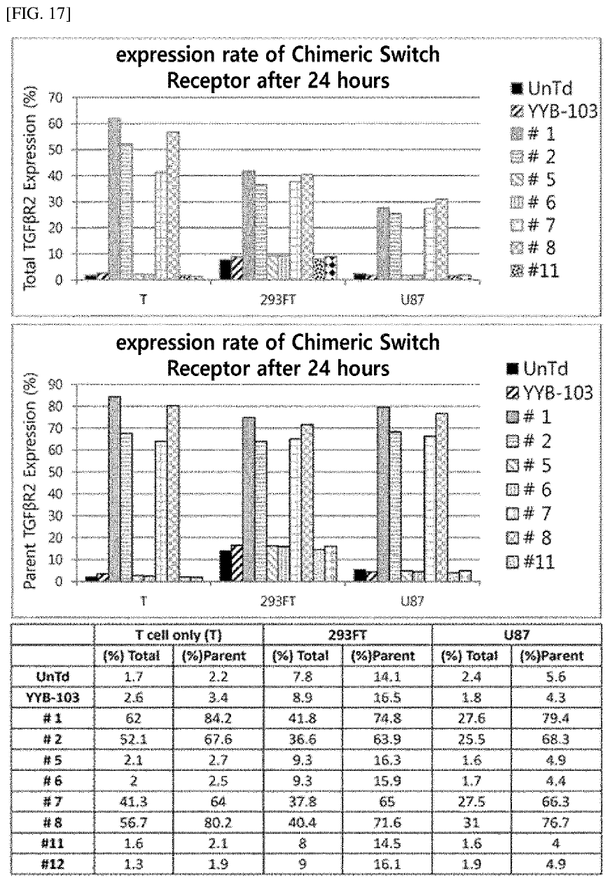

As a result of the analysis, as shown in FIG. 17, the expression of the chimeric switch receptors of TGFβR2 exodomain and transmembrane domain and an endodomain of IL-18R in the 4 groups (#1, #2, #7, and #8) after 24 hours of co-culture showed a tendency of a decrease in the groups co-cultured with human glioma cell line, U87 cells compared to those cultured with CAR-T cells only, but the average expression rate was 30% (FIG. 17). The expression of the chimeric switch receptors of TGFβR2 exodomain and transmembrane domain and an endodomain of IL-18R in the 4 groups (#1, #2, #7, and #8) after 48 hours of co-culture showed a tendency of a decrease in the groups co-cultured with U87 cells compared to those cultured with CAR-T cells only, but the average expression rate was 25%. The expression of the chimeric switch receptors of TGFβR2 exodomain and transmembrane domain and an endodomain of IL-18R in the 4 groups (#1, #2, #7, and #8) after 72 hours of co-culture showed a tendency of a decrease in the groups co-cultured with human glioma cell line, U87 cells compared to those cultured with CAR-T cells only, but the average expression rate was 15%. Although the changes in the expression rate were greater compared to one without co-culture with target cells or the co-culture with 293FT cells (which were used as the control group for normal cells), by the confirmation of the results of the chimeric switch receptor expression rate of TGFβR2 exodomain and transmembrane domain and an endodomain of IL-18R against human glioma cell line, U87 in which IL13Rα2 is overexpressed, the chimeric antigen receptor (CAR) according to the present invention is expected to show excellent in vivo persistence and anti-tumor effects of CAR-T cells in a hostile tumor microenvironment.

### Experimental Example 8: Checking of Cytokine (IFN-γ) Production by T Cells, which were Transformed with Novel Chimeric Antigen Receptors, Against Target Cells

Experimental Methods

In order to confirm the cytokine (IFN-γ) production after 24 hours and 48 hours of co-culture between the CAR-T cells prepared in Example 2 (#1, #2, #5, #6, #7, #8, #11, or #12) and human glioma cell line (U87 cells) and 293FT target cells (which were used as a control group for normal cells), the CAR-T cells (effector cells) in which the CAR expression rate is in the range of 20% to 40% were used 10 days after activation of the cells with an anti-CD3 Ab. The CAR-T cells were added to a 6-well plate at a ratio in which effector:target was 1×10⁶ cells:2×10⁶ cells, 6 mL of a culture medium was added per well, and the cells reacted at 37° C. for 24 hours. 100 μL of the supernatant was collected and transferred to a 1.5 mL tube, the cells were cultured further up to 48 hours, and 100 μL of the supernatant was collected from the culture in the same manner.

The IFN-γ analysis experiment was performed as follows according to the manufacturer's instructions of the ELISA analyzer (R&D systems). 3 mL of the calibrator diluent RD6-21 was added to an IFN-γ standard bottle and mixed in a shaker for 15 minutes, dispensed in an amount of 1 mL per 1.5 mL tube to prepare two “standard 1”s. 500 μL was taken from 1 mL of the “standard 1” dispensed and was subjected to serial dilution to prepare up to “standard 7”. In order to prepare blanks, 500 μL of the culture medium and 500 μL of the calibrator diluent RD6-21 were mixed.

In order to dilute the samples by 1/20, 190 μL of the calibrator diluent RD6-21 was added into each of new 1.5 mL tubes as many as the number of samples. The assay samples were each prepared to a total volume of 200 μL by collecting 10 μL of the supernatant of each sample. To prepare a wash buffer, 500 mL of distilled water and 20 mL of wash buffer concentrate were mixed well in a 500 mL storage bottle.

Assay diluent RD1-51 (blue dye) in an amount of 100 μL each was added to the IFN-γ microplate for each well of “standard 1” to “standard 7”, blank, and samples. After adding 100 μL each of the blank, standards, and samples prepared above, a plate sealer was attached thereto and reacted at room temperature for 2 hours. After the reaction, the reaction solution was discarded, and 400 μL of the prepared wash buffer was added thereto, and the wells were washed 4 times. 200 μL of IFN-γ conjugate was added to each well, a plate sealer was attached thereto, and the reaction was performed at room temperature for 2 hours. The reaction solution was discarded, and the wells were washed 4 times using 400 μL of the wash buffer. After mixing the color reagent A:B in a 1:1 ratio, the mixture was added in an amount of 200 μL per well, a plate sealer was attached thereto, the light was blocked with foil, and the reaction was performed at room temperature for 30 minutes. After the reaction, 50 μL of the stop solution was added to each well and measured at 450 nm within 30 minutes.

Experimental Results

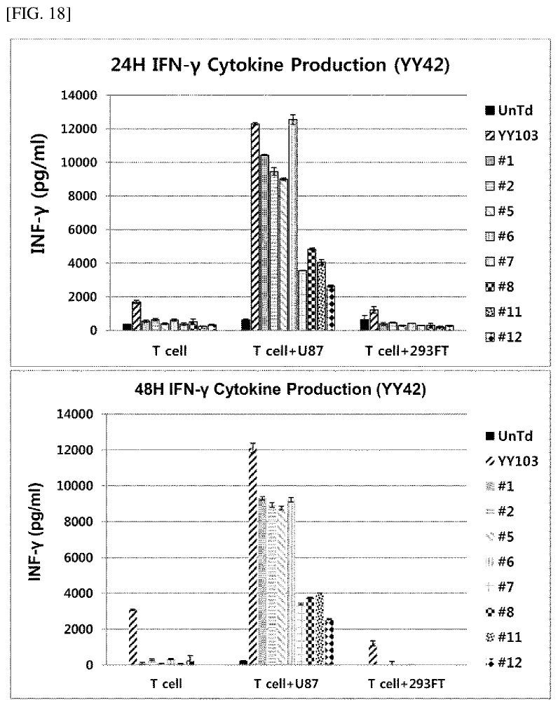

YYB103 was shown to secrete a relatively high amount of IFN-γ compared to other samples. When CAR-T cells were cultured in the presence of human glioma U87 cells, CAR-T (#1, #2, #5, and #6) containing an IL-7Rα signaling domain were shown to contain more IFN-γ compared to CAR-T (#7, #8, #11, and #12) containing an IL-2Rβ signaling domain. Results between samples after 24 hours and 48 hours of incubation showed similar patterns (FIG. 18).

As shown in FIG. 12, when the cytotoxicity was confirmed through LDH assay after 24 hours of co-culture between the CAR-T (which were transformed with the chimeric antigen receptor (CAR) of the present invention), U87 cells (human glioma cell line), and 293FT target cells (which are used as a control group for normal cells), the 8 groups showed similar abilities of killing target cells. However, considering that the production of IFN-γ was small and thus target cells could be killed without adverse effects caused by cytokines (cytokine release syndrome, CRS), in terms of safety, the effect of the CAR-T cell therapeutic is expected to have a fewer side effects while having excellent therapeutic effects.

### Experimental Example 9: Checking of Cytokine (IL-21) Production by CAR-T Cells, which were Transformed with Novel Chimeric Antigen Receptors

Experimental Methods

In order to confirm the cytokine (IL-21) production after 24 hours and 48 hours of co-culture between the CAR-T cells prepared in Example 2 (#1, #2, #5, #6, #7, #8, #11, or #12) and human glioma U87 cells and 293FT target cells (which were used as a control group for normal cells), the CAR-T cells (effector cells) in which the CAR expression rate is in the range of 20% to 40% were used 10 days after activation of the cells with an anti-CD3 Ab. The CAR-T cells were added to a 6-well plate at a ratio in which effector:target was 1×10⁶ cells:2×10⁶ cells, 6 mL of a culture medium was added per well, and the cells were reacted at 37° C. for 24 hours. Then, 100 μL of the supernatant was collected and transferred to a 1.5 mL tube, the cells were cultured further up to 48 hours, and 100 μL of the supernatant was collected from the culture in the same manner.

The IL-21 analysis experiment was performed as follows according to the manufacturer's instructions of the ELISA analyzer (Invitrogen). 100 μL of a capture antibody was added to each well of a 96-well ELISA plate and reacted at 4° C. overnight. After the reaction, the wells were washed 3 times using 250 μL of wash buffer. ELISA/ELISASPOT diluent was added in an amount of 200 μL per well and reacted at room temperature for one hour.

After the reaction, 100 μL each of the prepared blank, cytokine IL-21 standard, and samples were added thereto, and ELISA/ELISASPOT diluent was added in an amount of 100 μL per well, and a plate sealer was attached thereto and the reaction was performed at room temperature for two hours. After the reaction, the wells were washed 3 times using 250 μL of wash buffer. 100 μL of a detection antibody was added to each well, and a plate sealer was attached thereto and the reaction was performed at room temperature for one hour. After the reaction, the wells were washed 3 times using 250 μL of wash buffer. 100 μL of Avidin-HRP was added to each well, a plate sealer was attached thereto, and the reaction was performed at room temperature for 30 minutes. After the reaction, the wells were washed 5 times using 250 μL of wash buffer. 100 μL of 1×TMB solution was added to each well, a plate sealer was attached thereto, and the reaction was performed at room temperature for 30 minutes. After the reaction, 50 μL of the stop solution was added to each well and measured at 450 nm within 30 minutes.

Experimental Results

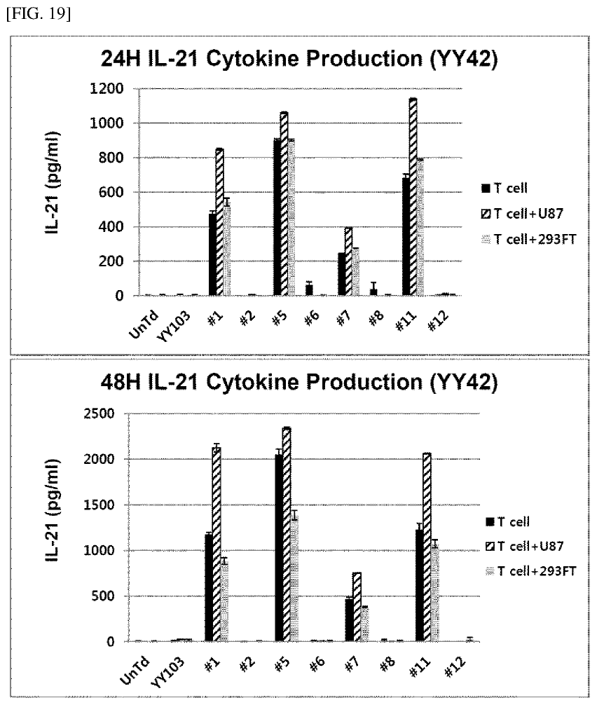

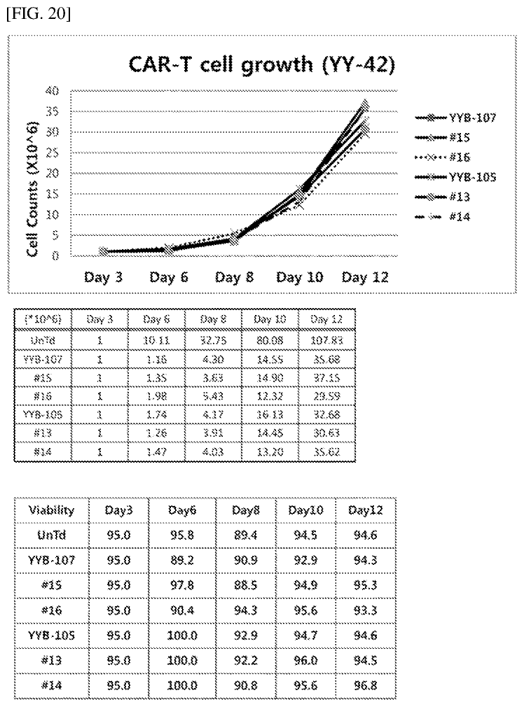

It was confirmed that IL-21 was secreted in IL-21 gene-containing CAR-T cells (#1, #5, #7, and #11). When the CAR-T cells were co-cultured with human glioma U87 cells, a greater amount of IL-21 was secreted, and considering that the overall experimental results at 24 hours and 48 hours were similar, no significant difference according to the incubation time was observed. As the result of the experiment at 48 hours showed a higher IL-21 concentration than that at 24 hours, the secretion of IL-21 appeared to proceed steadily (FIG. 19).

The secretion of IL-21 in an environment of cancer cells is speculated to increase the ability to kill cancer cells by assisting the activation of innate immune-related cells.

### Example 3: Preparation of Cancer Antigen EGFRvIII Targeting CAR Vector and aVβ3 Targeting CAR Vector

In the case of EGFRvIII, which is a major antigen for tumors such as glioblastoma and lung cancer, in order to reduce the side effects of CAR-T cells through non-specific binding while minimizing the binding affinity for EGFR wild-type in which the specificity for EGFRvIII is maintained, in SEQ ID NO: 33, positions 52 to 57 of SEQ ID NO: 3 were first changed from STGGYN to DPENDE (a CDR2 part of a heavy chain), position 101 of SEQ ID NO: 3 was changed from S to G (a CDR3 part of a heavy chain), and position 229 of SEQ ID NO: 3 was changed from V to G (a CDR3 part of a light chain).

Human CD3 (P20963-1), human CD8A (P01732), human 4-1BB (Q07011), human CD3Z (P20963), a Gaussia princeps luciferase, and human kappa light chain signal sequence (HuVHCAMP) were optimized using scientific literature and publicly available databases, and thereby chimeric antigen receptor-containing polypeptides consisting of codon-optimized synthetic DNA (#13, #14, #15, and #16 of SEQ ID NOS: 34 to 37) were prepared. The completed structure of CAR-containing polypeptide #13 includes a Kozak consensus ribosome-binding sequence, a CD8A signal sequence, an antigen binding domain which binds to EGFRvlll of SEQ ID NO: 3, three glycines (GGG) which are introduced between an antigen binding domain and a hinge region so as to increase the expression of a chimeric antigen receptor by increasing the solubility of a CAR protein, a hinge region of human CD8A, a human CD8 transmembrane domain, a cytoplasmic 4-1BB costimulatory signal domain, a CD3 cytoplasmic domain (SEQ ID NO: 18), and an XhoI/NotI cleavage site.

The finally prepared CAR gene fragment was conjugated to the MFG retrovirus expression vector digested with XhoI/NotI (Emtage P C et al., Clin Cancer Res, 2008, 14:8112-8122). In this Example, in order to compare the activity of chimeric antigen receptors, YYB105 (SEQ ID NO: 39; see PCT International Publication No. WO 2017/023138) was additionally prepared.

The completed structure of CAR-containing polypeptide #14 includes a Kozak consensus ribosome-binding sequence, a CD8A signal sequence, an antigen binding domain which binds to EGFRvlll of SEQ ID NO: 33, three glycines (GGG) which are introduced between an antigen binding domain and a hinge region so as to increase the expression of a chimeric antigen receptor by increasing the solubility of a CAR protein, a hinge region of human CD8A, a human CD8 transmembrane domain, a cytoplasmic 4-1BB costimulatory signal domain, a CD3ζ cytoplasmic domain (SEQ ID NO: 18), and an XhoI/NotI cleavage site. The finally prepared CAR gene fragment was conjugated to the MFG retrovirus expression vector digested with XhoI/NotI (Emtage P C et al., Clin Cancer Res, 2008, 14:8112-8122).

In the case of aVβ3 anti-angiogenic, the CAR expression rate and persistence in CAR-T cells targeting these were optimized. In the case of aVβ3, the CAR expression rate and persistence in CAR-T cells targeting these were optimized using Gaussia princeps luciferase signal sequence or CD8 signal sequence (SEQ ID NO: 36 and SEQ ID NO: 37).

The completed structure of CAR-containing polypeptide #15 includes a Kozak consensus ribosome-binding sequence, a Gaussia princeps luciferase signal sequence, SEQ ID NO: 5 (an antigen binding domain which binds to aVβ3), three glycines (GGG) which are introduced between an antigen binding domain and a hinge region so as to increase the expression of a chimeric antigen receptor by increasing the solubility of a CAR protein, a hinge region of human CD8A, a human CD8 transmembrane domain, a cytoplasmic 4-1BB costimulatory signal domain, a CD3ζ cytoplasmic domain (SEQ ID NO: 18), and an XhoI/NotI cleavage site. The finally prepared CAR gene fragment was conjugated to the MFG retrovirus expression vector digested with XhoI/NotI (Emtage P C et al., Clin Cancer Res, 2008, 14:8112-8122). In this Example, in order to compare the activity of chimeric antigen receptors, YYB107 (SEQ ID NO: 38; see PCT International Publication No. WO 2017/023138) was additionally prepared.

The completed structure of CAR-containing polypeptide #16 includes a Kozak consensus ribosome-binding sequence, a Gaussia princeps luciferase signal sequence, SEQ ID NO: 5 (an antigen binding domain which binds to aVβ3), three glycines (GGG) which are introduced between an antigen binding domain and a hinge region so as to increase the expression of a chimeric antigen receptor by increasing the solubility of a CAR protein, a hinge region of human CD8A, a human CD8 transmembrane domain, a cytoplasmic 4-1BB costimulatory signal domain, a CD3ζ cytoplasmic domain (SEQ ID NO: 18), and an XhoI/NotI cleavage site. The finally prepared CAR gene fragment was conjugated to the MFG retrovirus expression vector digested with XhoI/NotI (Emtage P C et al., Clin Cancer Res, 2008, 14:8112-8122).

### Example 4: Preparation of CAR-T Cells Transformed with Chimeric Antigen Receptors

High-titer CAR-expressing PG13 clones were prepared such that Phoenix-Ampho and Phoenix-Eco cells were transiently transfected with the retroviral expression vector prepared in Example 1, and then, cell-free vector stocks were prepared from the transfected Phoenix-Ampho and Phoenix-Eco cells by transfecting PG13 cells. For high-titer monoclones, PG13/#13, PG13/#14, PG13/#15, and PG13/#16 cells were stained using an anti-myc Ab (BD Pharmingen), and these monoclones were isolated using a flow cytometer. The transduction degree of PG13/#13, PG13/#14, PG13/#15, and PG13/#16 cells, which were transduced using an anti-myc Ab, was analyzed using a flow cytometer. The supernatants of the transduced PG13/#13, PG13/#14, PG13/#15, and PG13/#16 cells contained retrovirus, and the supernatants were collected for genetic modification of T cells. The peripheral blood mononuclear cells (PBMCs) were separated using centrifugation by adding the whole blood obtained from a healthy human donor into Ficoll Paque (GE Healthcare). The separated PBMCs were cultured by adding an anti-CD3 monoclonal antibody (eBioscience) at a concentration of 100 ng/mL under the condition of human IL-2 (Novartis) at a concentration of 100 IU/mL to activate the T cell fraction (BL Levine, Cancer Gene Therapy, 2015, 22:79-84). Two to three days after the cultivation, most of the cells were T cells and included natural killer cells at a percentage of 0% to 2%. Two to three days after the activation step, the T cells were subjected to transduction two times over two days using the retroviral supernatant and washed, and then proliferated for 14 days in a flask. IL-2 was maintained at a concentration of 100 IU/mL. The T cells modified as such were used for an analysis experiment.

### Experimental Example 1: Checking of Growth Rate and Viability of CAR-T Cells Transformed with Chimeric Antigen Receptors

Experimental Results

For the T cells prepared in Example 4 above, the number of cells was counted to confirm the growth rate and viability rate of CAR-T, and the results are shown in FIG. 20. The number of cells and growth rate of all of the groups (#13, #14, #15, and #16) were shown to be similar to those of the control group (i.e., YYB105) depending on the day of the week, the cells were shown to grow rapidly from Day 12 of culture, and the viability was also shown to be 90% or higher (see FIG. 20).

### Experimental Example 2: Checking of CAR Expression Rate on the Cell Surface of CAR-T Cells Transformed with Chimeric Antigen Receptors

Experimental Methods (Flow Cytometric Analysis)

For flow cytometry (>30,000 events), a BD LSRII device (Becton Dickinson) and BD FACSDiva software (Becton Dickinson) were used. Specifically, the cells were washed once with PBS containing 2% bovine serum albumin before adding a PE-conjugated anti-myc antibody (BD Pharmingen) thereto. After washing, the cells were reacted with each antibody at 4° C. for 30 minutes in a state where light was blocked and washed once, and thereafter, the expression rate of CAR on the surface of transduced T cells was checked.

Experimental Results

In order to confirm whether the 4 kinds of CAR prepared in Example 2 (#13, #14, #15, and #16) were expressed on the T cell surface, T cell cultivation was performed for 14 days according to Example 4, and then flow cytometric analysis was performed according to the experimental method.

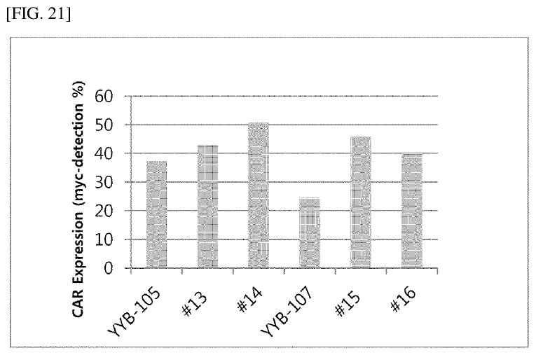

As a result of the analysis, as shown in FIG. 21, the expression rate of the chimeric antigen receptors expressed on the surface of live T cells was shown to increase in #13 (43.0%) and #14 (50.8%) compared to YYB105 (37.4%) (i.e., a control group) (FIG. 21); and was shown to increase in #15 (45.9%) and #16 (40.0%) compared to YYB107 (24.6%) (i.e., a control group) (FIG. 21).

### INDUSTRIAL APPLICABILITY

The present invention relates to rapidly developing a CAR-T cell in the field of cancer treatment. The CAR-T cell according to the present invention has a remarkably excellent expression rate and persistence, and thus has an improved therapeutic effect for solid cancer, etc., and can be effectively used in the field of customized cancer treatment.

[SEQUENCE LISTING]

SEQ ID NO: 1 {antigen binding wild type IL13 domain binding to IL13Ra2}
Length: 112
Type: ligand protein
Organism: human
Sequence:
G P V P P S T A L R E L I E E L V N I T Q N Q K A P L C N G S M V W S I N L T A G M Y
C A A L E S L I N V S G C S A I E K T Q R M L S G F C P H K V S A G Q F S S L H V R D
T K I E V A Q F V K D L L L H L K K L F R E G Q F N
SEQ ID NO: 2 {antigen binding domain capable of binding to an antigen associated with an
angiogenic activity }
Length: 92
Type: ligand protein
Organism: human
Sequence:
E V V A A T P T S L L I S W R H P H F P T R Y Y R I T Y G E T G G N S P V Q E F T V L Q
P P S T A T I S G L K P G V D Y T I T V Y A V V E R N G R E L N T P P I S I N Y R T H H
H H H H
SEQ ID NO: 3 {antigen binding domain binding to EGFRvIII}
Length: 252
Type: scFv protein
Organism: human
Sequence:
Q V Q L Q E S G G G L V K P G G S L K L S C A A S G F T F S K F G M S W V R Q T P D
K R L E W V A S I S T G G Y N T F Y S D N V K G R F T I S R D N A K N T L Y L Q M S S
L K S E D T A M Y Y C A R G Y S S T S F A M D Y W G Q G T M V T V S S G S T S G S G
K P G S G E G S D I Q M T Q S P S S L S A S V G D R V T I T C M T S T D I D D D M N W
Y Q Q K P G K T P K L L I Y E G N T L R P G V P S R F S G S G S G T D F I F T I S S L Q P
E D I A T Y Y C L Q S F N V P L T F G G G T K V E I K E Q K L I S E E D L
SEQ ID NO: 4 {antigen binding domain binding to EphA2}
Length: 141
Type: ligand protein
Organism: human
Sequence:
D R H T V F W N S S N P K F R N E D Y T I H V Q L N D Y V D I I C P H Y E D H S V A D
A A M E Q Y I L Y L V E H E E Y Q L C Q P Q S K D Q V R W Q C N R P S A K H G P E K
L S E K F Q R F T A F A L A K E F K A G H S Y Y Y I S K P I H Q H E D R C L R L K V T
V S G E Q K L I S E E D L
SEQ ID NO: 5 {antigen binding domain binding to αVβ3}
Length: 104
Type: ligand protein
Organism: human
Sequence:
V S D V P R D L E V V A A T P T S L L I S W D A P A V T V R Y Y R I T Y G E T G G N S
P V Q E F T V P G S K S T A T I S G L K P G V D Y T I T V Y A V T P R G D W N E G S K
P I S I N Y R T E Q K L I S E E D L
SEQ ID NO: 6 {antigen binding domain binding to glypican1}
Length: 418
Type: ligand protein
Organism: human
Sequence:
T S P C D N F D C Q N G A Q C I V R I N E P I C Q C L P G Y Q G E K C E K L V S V N F I
N K E S Y L Q I P S A K V R P Q T N I T L Q I A T D E D S G I L L Y K G D K D H I A V E
L Y R G R V R A S Y D T G S H P A S A I Y S V E T I N D G N F H I V E L L A L D Q S L S
L S V D G G N P K I I T N L S K Q S T L N F D S P L Y V G G M P G K S N V A S L R Q A
P G Q N G T S F H G C I R N L Y I N S E L Q D F Q K V P M Q T G I L P G C E P C H K K
V C A H G T C Q P S S Q A G F T C E C Q E G W M G P L C D Q R T N D P C L G N K C V
H G T C L P I N A F S Y S C K C L E G H G G V L C D E E E D L F N P C Q A I K C K H G
K C R L S G L G Q P Y C E C S S G Y T G D S C D R E I S C R G E R I R D Y Y Q K Q Q G
Y A A C Q T T K K V S R L E C R G G C A G G Q C C G P L R S K R R K Y S F E C T D G
S S F V D E V E K V V K C G C T R C V S E Q K L I S E E D L
SEQ ID NO: 7 {antigen binding domain binding to mesothelin}
Length: 262
Type: scFv protein
Organism: human
Sequence:
QVQLQESGPGLVKPSETLSLTCTVSGGSVSSGSYYWSWIRQPPGKGLEWIGYIYYSGSTN
YNPSLKSRVTISVDTSKNQFSLKLSSVTAADTAVYYCAREGKNGAFDIWGQGTMVTVSS
GSTSGSGKPGSGEGSQVQLQQSGPGLVTPSQTLSLTCAISGDSVSSNSATWNWIRQSPSR
GLEWLGRTYYRSKWYNDYAVSVKSRMSINPDTSKNQFSLQLNSVTP
EDTAVYYCARGMMTYYYGMDV WGQGTTVTVSSGILGS
SEQ ID NO: 8 {hinge region sequence −1}
Length: 47
Type: protein
Organism: human
Sequence:
K P T T T P A P R P P T P A P T I A S Q P L S L R P E A C R P A A G G A V H T R G L D F
A C D
SEQ ID NO: 9 {hinge region sequence −2}
Length: 45
Type: protein
Organism: human
Sequence:
K P T T T P A P R P P T P A P T I A S Q P L S L R P E A A R P A A G G A V H T R G L D F
A
SEQ ID NO: 10 {transmembrane domain sequence −1}
Length: 21
Type: protein
Organism: human
Sequence:
I Y I W A P L A G T C G V L L L S L V I T
SEQ ID NO: 11 {transmembrane domain sequence −2}
Length: 23
Type: protein
Organism: human
Sequence:
L A Y L L D G I L F I Y G V I L T A L F L R V
SEQ ID NO: 12 {4-1BB}
Length: 42
Type: protein
Organism: human
Sequence:
K R G R K K L L Y I F K Q P F M R P V Q T T Q E E D G C S C R F P E E E E G G C E L
SEQ ID NO: 13 {Wild type CD28}
Length: 41
Type: protein
Organism: human
Sequence:
R S K R S R L L H S D Y M N M T P R R P G P T R K H Y Q P Y A P P R D F A A Y R S
SEQ ID NO: 14 {IL-7Rα}
Length: 459
Type: protein
Organism: human
Sequence:
MTILGTTFGMVFSLLQVVSGESGYAQNGDLEDAELDDYSFSCYSQLEVNGSQHSLTCAF
EDPDVNITNLEFEICGALVEVKCLNFRKLQEIYFIETKKFLLIGKSNICVKVGEKSLTCKKI
DLTTIVKPEAPFDLSVVYREGANDFVVTFNTSHLQKKYVKVLMHDVAYRQEKDENKW
THVNLSSTKLTLLQRKLQPAAMYEIKVRSIPDHYFKGFWSEWSPSYYFRTPEINNSSGEM
DPILLTISILSFFSVALLVILACVLWKKRIKPIVWPSLPDHKKTLEHLCKKPRKNLNVSFNP
ESFLDCQIHRVDDIQARDEVEGFLQDTFPQQLEESEKQRLGGDVQSPNCPSEDVVITPESF
GRDSSLTCLAGNVSACDAPILSSSRSLDCRESGKNGPHVYQDLLLSLGTTNSTLPPPFSLQ
SGILTLNPV AQGQPILTSLGSNQEEAYVTMSSFYQNQ
SEQ ID NO: 15 {part of IL-7Rα}
Length: 64
Type: protein
Organism: human
Sequence:
KKRIKPIVWPSLPDHKKTLEHLCKKPRKNLNVSFNPESFLDCQIHRVDDIQARDEVEGFL
QDTF
SEQ ID NO: 16 {IL-2Rβ}
Length: 551
Type: protein
Organism: human
Sequence:
MAAPALSWRLPLLILLLPLATSWASAAVNGTSQFTCFYNSRANISCVWSQDGALQDTSC
QVHAWPDRRRWNQTCELLPVSQASWACNLILGAPDSQKLTTVDIVTLRVLCREGVRWR
VMAIQDFKPFENLRLMAPISLQNVHVETHRCNISWEISQASHYFERHLEFEARTLSPGHT
WEEAPLLTLKQKQEWICLETLTPDTQYEFQVRVKPLQGEFTTWSPWSQPLAFRTKPAAL
GKDTIPWLGHLLVGLSGAFGFIILVYLLINCRNTGPWLKKVLKCNTPDPSKFFSQLSSEH
GGDVQKWLSSPFPSSSFSPGGLAPEISPLEVLERDKVTQLLLQQDKVPEPASLSSNHSLTS
CFTNQGYFFFHLPDALEIEACQVYFTYDPYSEEDPDEGVAGAPTGSSPQPLQPLSGEDDA
YCTFPSRDDLLLFSPSLLGGPSPPSTAPGGSGAGEERMPPSLQERVPRDWDPQPLGPPTPG
VPDLVDFQPPPELVLREAGEEVPDAGPREGVSFPWSRPPGQGEFRALNARLPLNTD
AYLSLQELQGQDPTHLV
SEQ ID NO: 17 {part of IL-2Rβ}
Length: 104
Type: protein
Organism: human
Sequence:
NCRNTGPWLKKVLKCNTPDPSKFFSQLSSEHGGDVQKWLSSPFPSSSFSPGGLAPEISPLE
VLERDKVTQLLLQQDKVPEPASLSSNHSLTSCFTNQGYFFFHL
SEQ ID NO: 18 {CD3ζ}
Length: 113
Type: protein
Organism: human
Sequence:
R V K F S R S A D A P A Y Q Q G Q N Q L Y N E L N L G R R E E Y D V L D K R R G R D
P E M G G K P Q R R K N P Q E G L Y N E L Q K D K M A E A Y S E I G M K G E R R R G
K G H D G L Y Q G L S T A T K D T Y D A L H M Q A L P P R
SEQ ID NO: 19 {TGF-βR2 exodomain}
Length: 137
Type: protein
Organism: human
Sequence:
TIPPHVQKSVNNDMIVTDNNGAVKFPQLCKFCDVRFSTCDNQKSCMSNCSITSICEKPQE
VCVAVWRKNDENITLETVCHDPKLPYHDFILEDAASPKCIMKEKKKPGETFFMCSCSSD
ECNDNIIFSEEYNTSNPD
SEQ ID NO: 20 {transmembrane domain and endodomain of IL-18R}
Length: 219
Type: protein
Organism: human
Sequence:
PGHVFTRGMIIAVLILVAVVCLVTVCVIYRVDLVLFYRHLTRRDETLTDGKTYDAFVSY
LKECRPENGEEHTFAVEILPRVLEKHFGYKLCIFERDVVPGGAVVDEIHSLIEKSRRLIIVL
SKSYMSNEVRYELESGLHEALVERKIKIILIEFTPVTDFTFL
PQSLKLLKSHRVLKWKADKSLSYNSRFWKNLLYLMPAKTVKPGRDEPEVLPV LSES
SEQ ID NO: 21 {IL-21}
Length: 133
Type: protein
Organism: human
Sequence:
QGQDRHMIRMRQLIDIVDQLKNYVNDLVPEFLPAPEDVETNCEWSAFSCFQKAQLKSA
NTGNNERIINVSIKKLKRKPPSTNAGRRQKHRLTCPSCDSYEKKPPK
EFLERFKSLLQKMIHQHLSSRTHGSEDS
SEQ ID NO: 22 {YYB 103}
Length: 359
Type: protein
Organism: human
Sequence:
M G W S C I I L F L V A T A T G V H S G P V P P S T A L R K L I E E L V N I T Q N Q K A
P L C N G S M V W S I N L T A G M Y C A A L E S L I N V S G C S A I E K T Q D M L D G
F C P H K V S A G Q F S S L H V R D T K I E V A Q F V K D L L L H L K K L F K E G Q F
N G G G P R K P T T T P A P R P P T P A P T I A S Q P L S L R P E A C R P A A G G A V H
T R G L D F A C D I Y I W A P L A G T C G V L L L S L V I T K R G R K K L L Y I F K Q P
F M R P V Q T T Q E E D G C S C R F P E E E E G G C E L R V K F S R S A D A P A Y Q Q
G Q N Q L Y N E L N L G R R E E Y D V L D K R R G R D P E M G G K P Q R R K N P Q E
G L Y N E L Q K D K M A E A Y S E I G M K G E R R R G K G H D G L Y Q G L S T A T K
D T Y D A L H M Q A L P P R
SEQ ID NO: 23 {#1}
Length: 998
Type: protein
Organism: human
Sequence:
MGWSCIILFLVATATGVHSGPVPPSTALRKLIEELVNITQNQKAPLCNGSMVWSINLTAG
MYCAALESLINVSGCSAIEKTQDMLDGFCPHKVSAGQFSSLHVRDTKIEVAQFVKDLLL
HLKKLFKEGQFNGGGPRKPTTTPAPRPPTPAPTIASQPLSLRPEACRPAAGGAVHTRGLD
FACDIYIWAPLAGTCGVLLLSLVITKRGRKKLLYIFKQPFMRPVQTTQEEDGCSCRFPEE
EEGGCELKKRIKPIVWPSLPDHKKTLEHLCKKPRKNLNVSFNPESFLDCQIHRVDDIQAR
DEVEGFLQDTFRVKFSRSADAPAYQQGQNQLYNELNLGRREEYDVLDKRRGRDPEMG
GKPQRRKNPQEGLYNELQKDKMAEAYSEIGMKGERRRGKGHDGLYLPQSTATKDTYD
YVTMQALPPREGRGSLLTCGDVEENPGPMALPVTALLLPLALLLHAARPTIPPHVQKSV
NNDMIVTDNNGAVKFPQLCKFCDVRFSTCDNQKSCMSNCSITSICEKPQEVCVAVWRK
NDENITLETVCHDPKLPYHDFILEDAASPKCIMKEKKKPGETFFMCSCSSDECNDNIIFSE
EYNTSNPDPGHVFTRGMIIAVLILVAVVCLVTVCVIYRVDLVLFYRHLTRRDETLTDGKT
YDAFVSYLKECRPENGEEHTFAVEILPRVLEKHFGYKLCIFERDVVPGGAVVDEIHSLIE
KSRRLIIVLSKSYMSNEVRYELESGLHEALVERKIKIILIEFTPVTDFTFLPQSLKLLKSHRV
LKWKADKSLSYNSRFWKNLLYLMPAKTVKPGRDEPEVLPVLSESRRKRSGSGATNFSL
LKQAGDVEENPGPMYRMQLLSCIALSLALVTNSQGQDRHMIRMRQLIDIVDQLKNYVN
DLVPEFLPAPEDVETNCEWSAFSCFQKAQLKSANTGNNERIINVSIKKLKRKPPSTNAGR
RQKHRLTCPSCDSYEKKPPKEFLERFKSLLQKMIHQHLSSRTHGSEDS
SEQ ID NO: 24 {#2}
Length: 818
Type: protein
Organism: human
Sequence:
MGWSCIILFLVATATGVHSGPVPPSTALRKLIEELVNITQNQKAPLCNGSMVWSINLTAG
MYCAALESLINVSGCSAIEKTQDMLDGFCPHKVSAGQFSSLHVRDTKIEVAQFVKDLLL
HLKKLFKEGQFNGGGPRKPTTTPAPRPPTPAPTIASQPLSLRPEACRPAAGGAVHTRGLD
FACDIYIWAPLAGTCGVLLLSLVITKRGRKKLLYIFKQPFMRPVQTTQEEDGCSCRFPEE
EEGGCELKKRIKPIVWPSLPDHKKTLEHLCKKPRKNLNVSFNPESFLDCQIHRVDDIQAR
DEVEGFLQDTFRVKFSRSADAPAYQQGQNQLYNELNLGRREEYDVLDKRRGRDPEMG
GKPQRRKNPQEGLYNELQKDKMAEAYSEIGMKGERRRGKGHDGLYLPQSTATKDTYD
YVTMQALPPREGRGSLLTCGDVEENPGPMALPVTALLLPLALLLHAARPTIPPHVQKSV
NNDMIVTDNNGAVKFPQLCKFCDVRFSTCDNQKSCMSNCSITSICEKPQEVCVAVWRK
NDENITLETVCHDPKLPYHDFILEDAASPKCIMKEKKKPGETFFMCSCSSDECNDNIIFSE
EYNTSNPDPGHVFTRGMIIAVLILVAVVCLVTVCVIYRVDLVLFYRHLTRRDETLTDGKT
YDAFVSYLKECRPENGEEHTFAVEILPRVLEKHFGYKLCIFERDVVPGGAVVDEIHSLIE
KSRRLIIVLSKSYMSNEVRYELESGLHEALVERKIKIILIEFTPVTDFTFLPQSLKLLKSHRV
LKWKADKSLSYNSRFWKNLLYLMPAKT VKPGRDEPEVLPVLSES
SEQ ID NO: 25 {#5}
Length: 594
Type: protein
Organism: human
Sequence:
MGWSCIILFLVATATGVHSGPVPPSTALRKLIEELVNITQNQKAPLCNGSMVWSINLTAG
MYCAALESLINVSGCSAIEKTQDMLDGFCPHKVSAGQFSSLHVRDTKIEVAQFVKDLLL
HLKKLFKEGQFNGGGPRKPTTTPAPRPPTPAPTIASQPLSLRPEACRPAAGGAVHTRGLD
FACDIYIWAPLAGTCGVLLLSLVITKRGRKKLLYIFKQPFMRPVQTTQEEDGCSCRFPEE
EEGGCELKKRIKPIVWPSLPDHKKTLEHLCKKPRKNLNVSFNPESFLDCQIHRVDDIQAR
DEVEGFLQDTFRVKFSRSADAPAYQQGQNQLYNELNLGRREEYDVLDKRRGRDPEMG
GKPQRRKNPQEGLYNELQKDKMAEAYSEIGMKGERRRGKGHDGLYLPQSTATKDTYD
YVTMQALPPREGRGSLLTCGDVEENPGPMYRMQLLSCIALSLALVTNSQGQDRHMIRM
RQLIDIVDQLKNYVNDLVPEFLPAPEDVETNCEWSAFSCFQKAQLKSANTGNNERIINVS
IKKLKRKPPSTNAGRRQKHRLTCPSCDSYEKKPPKEFLERFKSLLQKMIHQH
LSSRTHGSEDS
SEQ ID NO: 26 {#6}
Length: 423
Type: protein
Organism: human
Sequence:
MGWSCIILFLVATATGVHSGPVPPSTALRKLIEELVNITQNQKAPLCNGSMVWSINLTAG
MYCAALESLINVSGCSAIEKTQDMLDGFCPHKVSAGQFSSLHVRDTKIEVAQFVKDLLL
HLKKLFKEGQFNGGGPRKPTTTPAPRPPTPAPTIASQPLSLRPEACRPAAGGAVHTRGLD
FACDIYIWAPLAGTCGVLLLSLVITKRGRKKLLYIFKQPFMRPVQTTQEEDGCSCRFPEE
EEGGCELKKRIKPIVWPSLPDHKKTLEHLCKKPRKNLNVSFNPESFLDCQIHRVDDIQAR
DEVEGFLQDTFRVKFSRSADAPAYQQGQNQLYNELNLGRREEYDVLDKRRGRDPEMG
GKPQRRKNPQEGLYNELQKDKMAEAYSEIGMKGERRRGKGHDGLYLPQSTATKDTYD
YVTMQALPPR
SEQ ID NO: 27 {#7}
Length: 1038
Type: protein
Organism: human
Sequence:
MGWSCIILFLVATATGVHSGPVPPSTALRKLIEELVNITQNQKAPLCNGSMVWSINLTAG
MYCAALESLINVSGCSAIEKTQDMLDGFCPHKVSAGQFSSLHVRDTKIEVAQFVKDLLL
HLKKLFKEGQFNGGGPRKPTTTPAPRPPTPAPTIASQPLSLRPEACRPAAGGAVHTRGLD
FACDIYIWAPLAGTCGVLLLSLVITKRGRKKLLYIFKQPFMRPVQTTQEEDGCSCRFPEE
EEGGCELNCRNTGPWLKKVLKCNTPDPSKFFSQLSSEHGGDVQKWLSSPFPSSSFSPGGL
APEISPLEVLERDKVTQLLLQQDKVPEPASLSSNHSLTSCFTNQGYFFFHLRVKFSRSADA
PAYQQGQNQLYNELNLGRREEYDVLDKRRGRDPEMGGKPQRRKNPQEGLYNELQKDK
MAEAYSEIGMKGERRRGKGHDGLYLSLSTATKDTYLPQHMQALPPREGRGSLLTCGDV
EENPGPMALPVTALLLPLALLLHAARPTIPPHVQKSVNNDMIVTDNNGAVKFPQLCKFC
DVRFSTCDNQKSCMSNCSITSICEKPQEVCVAVWRKNDENITLETVCHDPKLPYHDFILE
DAASPKCIMKEKKKPGETFFMCSCSSDECNDNIIFSEEYNTSNPDPGHVFTRGMIIAVLIL
VAVVCLVTVCVIYRVDLVLFYRHLTRRDETLTDGKTYDAFVSYLKECRPENGEEHTFA
VEILPRVLEKHFGYKLCIFERDVVPGGAVVDEIHSLIEKSRRLIIVLSKSYMSNEVRYELES
GLHEALVERKIKIILIEFTPVTDFTFLPQSLKLLKSHRVLKWKADKSLSYNSRFWKNLLYL
MPAKTVKPGRDEPEVLPVLSESRRKRSGSGATNFSLLKQAGDVEENPGPMYRMQLLSCI
ALSLALVTNSQGQDRHMIRMRQLIDIVDQLKNYVNDLVPEFLPAPEDVETNCEWSAFSC
FQKAQLKSANTGNNERIINVSIKKLKRKPPSTNAGRRQKHRLTCPSCDSYEKKPPKEFLE
RFKSLLQKMIHQHLSSRTHGSEDS
SEQ ID NO: 28 {#8}
Length: 858
Type: protein
Organism: human
Sequence:
MGWSCIILFLVATATGVHSGPVPPSTALRKLIEELVNITQNQKAPLCNGSMVWSINLTAG
MYCAALESLINVSGCSAIEKTQDMLDGFCPHKVSAGQFSSLHVRDTKIEVAQFVKDLLL
HLKKLFKEGQFNGGGPRKPTTTPAPRPPTPAPTIASQPLSLRPEACRPAAGGAVHTRGLD
FACDIYIWAPLAGTCGVLLLSLVITKRGRKKLLYIFKQPFMRPVQTTQEEDGCSCRFPEE
EEGGCELNCRNTGPWLKKVLKCNTPDPSKFFSQLSSEHGGDVQKWLSSPFPSSSFSPGGL
APEISPLEVLERDKVTQLLLQQDKVPEPASLSSNHSLTSCFTNQGYFFFHLRVKFSRSADA
PAYQQGQNQLYNELNLGRREEYDVLDKRRGRDPEMGGKPQRRKNPQEGLYNELQKDK
MAEAYSEIGMKGERRRGKGHDGLYLSLSTATKDTYLPQHMQALPPREGRGSLLTCGDV
EENPGPMALPVTALLLPLALLLHAARPTIPPHVQKSVNNDMIVTDNNGAVKFPQLCKFC
DVRFSTCDNQKSCMSNCSITSICEKPQEVCVAVWRKNDENITLETVCHDPKLPYHDFILE
DAASPKCIMKEKKKPGETFFMCSCSSDECNDNIIFSEEYNTSNPDPGHVFTRGMIIAVLIL
VAVVCLVTVCVIYRVDLVLFYRHLTRRDETLTDGKTYDAFVSYLKECRPENGEEHTFA
VEILPRVLEKHFGYKLCIFERDVVPGGAVVDEIHSLIEKSRRLIIVLSKSYMSNEVRYELES
GLHEALVERKIKIILIEFTPVTDFTFLPQSLKLLKSHRVLKWKADKSLSYNSRFWKNLLYL
MPAKTVKPGRDEPEVLPVLSES
SEQ ID NO: 29 {#11}
Length: 634
Type: protein
Organism: human
Sequence:
MGWSCIILFLVATATGVHSGPVPPSTALRKLIEELVNITQNQKAPLCNGSMVWSINLTAG
MYCAALESLINVSGCSAIEKTQDMLDGFCPHKVSAGQFSSLHVRDTKIEVAQFVKDLLL
HLKKLFKEGQFNGGGPRKPTTTPAPRPPTPAPTIASQPLSLRPEACRPAAGGAVHTRGLD
FACDIYIWAPLAGTCGVLLLSLVITKRGRKKLLYIFKQPFMRPVQTTQEEDGCSCRFPEE
EEGGCELNCRNTGPWLKKVLKCNTPDPSKFFSQLSSEHGGDVQKWLSSPFPSSSFSPGGL
APEISPLEVLERDKVTQLLLQQDKVPEPASLSSNHSLTSCFTNQGYFFFHLRVKFSRSADA
PAYQQGQNQLYNELNLGRREEYDVLDKRRGRDPEMGGKPQRRKNPQEGLYNELQKDK
MAEAYSEIGMKGERRRGKGHDGLYLSLSTATKDTYLPQHMQALPPREGRGSLLTCGDV
EENPGPMYRMQLLSCIALSLALVTNSQGQDRHMIRMRQLIDIVDQLKNYVNDLVPEFLP
APEDVETNCEWSAFSCFQKAQLKSANTGNNERIINVSIKKLKRKPPSTNAGRRQKHRLT
CPSCDSYEKKPPKEFLERFKSLLQKMIHQHLSSRTHGSEDS
SEQ ID NO: 30 {#12}
Length: 463
Type: protein
Organism: human
Sequence:
MGWSCIILFLVATATGVHSGPVPPSTALRKLIEELVNITQNQKAPLCNGSMVWSINLTAG
MYCAALESLINVSGCSAIEKTQDMLDGFCPHKVSAGQFSSLHVRDTKIEVAQFVKDLLL
HLKKLFKEGQFNGGGPRKPTTTPAPRPPTPAPTIASQPLSLRPEACRPAAGGAVHTRGLD
FACDIYIWAPLAGTCGVLLLSLVITKRGRKKLLYIFKQPFMRPVQTTQEEDGCSCRFPEE
EEGGCELNCRNTGPWLKKVLKCNTPDPSKFFSQLSSEHGGDVQKWLSSPFPSSSFSPGGL
APEISPLEVLERDKVTQLLLQQDKVPEPASLSSNHSLTSCFTNQGYFFFHLRVKFSRSADA
PAYQQGQNQLYNELNLGRREEYDVLDKRRGRDPEMGGKPQRRKNPQEGLYNELQKDK
MAEAYSEIGMKGERRRGKGHDGLYLSLSTATKDTYLPQHMQALPPR
SEQ ID NO: 31 {mutated 3rd ITAM-1}
Length: 23
Type: protein
Organism: human
Sequence:
Y L P Q S T A T K D T Y D Y V T M Q A L P P R
SEQ ID NO: 32 {mutated 3rd ITAM-2}
Length: 23
Type: protein
Organism: human
Sequence:
Y L S L S T A T K D T Y L P Q H M Q A L P P R
SEQ ID NO: 33 {antigen binding domain binding to EGFRvIII}
Length: 252
Type: scFv protein
Organism: human
Sequence:
Q V Q L Q E S G G G L V K P G G S L K L S C A A S G F T F S K F G M S W V R Q T P D
K R L E W V A S I D P E N D E T F Y S D N V K G R F T I S R D N A K N T L Y L Q M S S
L K S E D T A M Y Y C A R G Y G S T S F A M D Y W G Q G T M V T V S S G S T S G S G
K P G S G E G S D I Q M T Q S P S S L S A S V G D R V T I T C M T S T D I D D D M N W
Y Q Q K P G K T P K L L I Y E G N T L R P G V P S R F S G S G S G T D F I F T I S S L Q P
E D I A T Y Y C L Q S F N G P L T F G G G T K V E I K E Q K L I S E E D L
SEQ ID NO: 34 {#13}
Length: 499
Type: protein
Organism: human
Sequence:
M A L P V T A L L L P L A L L L H A A R P Q V Q L Q E S G G G L V K P G G S L K L S C
A A S G F T F S K F G M S W V R Q T P D K R L E W V A S I S T G G Y N T F Y S D N V
K G R F T I S R D N A K N T L Y L Q M S S L K S E D T A M Y Y C A R G Y S S T S F A
M D Y W G Q G T M V T V S S G S T S G S G K P G S G E G S D I Q M T Q S P S S L S A S
V G D R V T I T C M T S T D I D D D M N W Y Q Q K P G K T P K L L I Y E G N T L R P G
V P S R F S G S G S G T D F I F T I S S L Q P E D I A T Y Y C L Q S F N V P L T F G G G T
K V E I K E Q K L I S E E D L G G G P R K P T T T P A P R P P T P A P T I A S Q P L S L R
P E A A R P A A G G A V H T R G L D F A L A Y L L D G I L F I Y G V I L T A L F L R V
K R G R K K L L Y I F K Q P F M R P V Q T T Q E E D G C S C R F P E E E E G G C E L K
F S R S A D A P A Y Q Q G Q N Q L Y N E L N L G R R E E Y D V L D K R R G R D P E M
G G K P Q R R K N P Q E G L Y N E L Q K D K M A E A Y S E I G M K G E R R R G K G H
D G L Y Q G L S T A T K D T Y D A L H M Q A L P P R
SEQ ID NO: 35 {#14}
Length: 499
Type: protein
Organism: human
Sequence:
M A L P V T A L L L P L A L L L H A A R P Q V Q L Q E S G G G L V K P G G S L K L S C
A A S G F T F S K F G M S W V R Q T P D K R L E W V A S I D P E N D E T F Y S D N V
K G R F T I S R D N A K N T L Y L Q M S S L K S E D T A M Y Y C A R G Y G S T S F A
M D Y W G Q G T M V T V S S G S T S G S G K P G S G E G S D I Q M T Q S P S S L S A S
V G D R V T I T C M T S T D I D D D M N W Y Q Q K P G K T P K L L I Y E G N T L R P G
V P S R F S G S G S G T D F I F T I S S L Q P E D I A T Y Y C L Q S F N G P L T F G G G T
K V E I K E Q K L I S E E D L G G G P R K P T T T P A P R P P T P A P T I A S Q P L S L R
P E A A R P A A G G A V H T R G L D F A L A Y L L D G I L F I Y G V I L T A L F L R V
K R G R K K L L Y I F K Q P F M R P V Q T T Q E E D G C S C R F P E E E E G G C E L K
F S R S A D A P A Y Q Q G Q N Q L Y N E L N L G R R E E Y D V L D K R R G R D P E M
G G K P Q R R K N P Q E G L Y N E L Q K D K M A E A Y S E I G M K G E R R R G K G H
D G L Y Q G L S T A T K D T Y D A L H M Q A L P P R
SEQ ID NO: 36 {#15}
Length: 349
Type: protein
Organism: human
Sequence:
M G V K V L F A L I C I A V A E A V S D V P R D L E V V A A T P T S L L I S W D A P A
V T V R Y Y R I T Y G E T G G N S P V Q E F T V P G S K S T A T I S G L K P G V D Y T I
T V Y A V T P R G D W N E G S K P I S I N Y R T E Q K L I S E E D L G G G P R K P T T T
P A P R P P T P A P T I A S Q P L S L R P E A C R P A A G G A V H T R G L D F A C D I Y
I W A P L A G T C G V L L L S L V I T K R G R K K L L Y I F K Q P F M R P V Q T T Q E
E D G C S C R F P E E E E G G C E L R V K F S R S A D A P A Y Q Q G Q N Q L Y N E L N
L G R R E E Y D V L D K R R G R D P E M G G K P Q R R K N P Q E G L Y N E L Q K D K
M A E A Y S E I G M K G E R R R G K G H D G L Y Q G L S T A T K D T Y D A L H M Q
A L P P R
SEQ ID NO: 37 {#16}
Length: 353
Type: protein
Organism: human
Sequence:
M A L P V T A L L L P L A L L L H A A R P V S D V P R D L E V V A A T P T S L L I S W
D A P A V T V R Y Y R I T Y G E T G G N S P V Q E F T V P G S K S T A T I S G L K P G
V D Y T I T V Y A V T P R G D W N E G S K P I S I N Y R T E Q K L I S E E D L G G G P R
K P T T T P A P R P P T P A P T I A S Q P L S L R P E A C R P A A G G A V H T R G L D F
A C D I Y I W A P L A G T C G V L L L S L V I T K R G R K K L L Y I F K Q P F M R P V
Q T T Q E E D G C S C R F P E E E E G G C E L R V K F S R S A D A P A Y Q Q G Q N Q L
Y N E L N L G R R E E Y D V L D K R R G R D P E M G G K P Q R R K N P Q E G L Y N E
L Q K D K M A E A Y S E I G M K G E R R R G K G H D G L Y Q G L S T A T K D T Y D
A L H M Q A L P P R
SEQ ID NO: 38 { YYB 107}
Length: 351
Type: protein
Organism: human
Sequence:
M G W S C I I L F L V A T A T G V H S V S D V P R D L E V V A A T P T S L L I S W D A
P A V T V R Y Y R I T Y G E T G G N S P V Q E F T V P G S K S T A T I S G L K P G V D
Y T I T V Y A V T P R G D W N E G S K P I S I N Y R T E Q K L I S E E D L G G G P R K P
T T T P A P R P P T P A P T I A S Q P L S L R P E A C R P A A G G A V H T R G L D F A C
D I Y I W A P L A G T C G V L L L S L V I T K R G R K K L L Y I F K Q P F M R P V Q T T
Q E E D G C S C R F P E E E E G G C E L R V K F S R S A D A P A Y Q Q G Q N Q L Y N E
L N L G R R E E Y D V L D K R R G R D P E M G G K P Q R R K N P Q E G L Y N E L Q K
D K M A E A Y S E I G M K G E R R R G K G H D G L Y Q G L S T A T K D T Y D A L H
M Q A L P P R
SEQ ID NO: 39 {YYB 105}
Length: 497
Type: protein
Organism: human
Sequence:
M G W S C I I L F L V A T A T G V H S Q V Q L Q E S G G G L V K P G G S L K L S C A A
S G F T F S K F G M S W V R Q T P D K R L E W V A S I S T G G Y N T F Y S D N V K G
R F T I S R D N A K N T L Y L Q M S S L K S E D T A M Y Y C A R G Y S S T S F A M D
Y W G Q G T M V T V S S G S T S G S G K P G S G E G S D I Q M T Q S P S S L S A S V G
D R V T I T C M T S T D I D D D M N W Y Q Q K P G K T P K L L I Y E G N T L R P G V P
S R F S G S G S G T D F I F T I S S L Q P E D I A T Y Y C L Q S F N V P L T F G G G T K V
E I K E Q K L I S E E D L G G G P R K P T T T P A P R P P T P A P T I A S Q P L S L R P E
A A R P A A G G A V H T R G L D F A L A Y L L D G I L F I Y G V I L T A L F L R V K R
G R K K L L Y I F K Q P F M R P V Q T T Q E E D G C S C R F P E E E E G G C E L K F S
R S A D A P A Y Q Q G Q N Q L Y N E L N L G R R E E Y D V L D K R R G R D P E M G
G K P Q R R K N P Q E G L Y N E L Q K D K M A E A Y S E I G M K G E R R R G K G H D
G L Y Q G L S T A T K D T Y D A L H M Q A L P P R
SEQ ID NO: 40 {T2A peptide}
Length: 21
Type: protein
Organism: human
Sequence:
G S G E G R G S L L T C G D V E E N P G P
SEQ ID NO: 41 {P2A peptide}
Length: 22
Type: protein
Organism: human
Sequence:
G S G A T N F S L L K Q A G D V E E N P G P
SEQ ID NO: 42 {E2A peptide}
Length: 23
Type: protein
Organism: human
Sequence:
G S G Q C T N Y A L L K L A G D V E S N P G P
SEQ ID NO: 43 {F2A peptide}
Length: 25
Type: protein
Organism: human
Sequence:
G S G V K Q T L N F D L L K L A G D V E S N P G P

1. A polypeptide comprising a chimeric antigen receptor, which comprises an antigen binding domain; a hinge region; a transmembrane domain; a costimulatory domain; and a signaling domain,
wherein the signaling domain comprises a CD3ζ domain.

2. The polypeptide of claim 1, which comprises IL-7Rα or a part thereof interposed between the costimulatory domain and the CD3ζ domain.

3. The polypeptide of claim 1, which comprises IL-2Rβ or a part thereof interposed between the costimulatory domain and the CD3ζ domain.

4. The polypeptide of claim 2, wherein a part of IL-7Rα is a sequence of SEQ ID NO: 15.

5. The polypeptide of claim 3, wherein a part of IL-2Rβ is a sequence of SEQ ID NO: 17.

6. The polypeptide of claim 2, which comprises three immunoreceptor tyrosine-based activation motifs (ITAMs) within the CD3ζ domain,
wherein in a third ITAM region, a first motif, YxxL, is substituted with YxxQ; and a second motif region, YxxLHM, is substituted with YxYVTM.

7. The polypeptide of claim 3, which comprises three immunoreceptor tyrosine-based activation motifs (ITAMs) interposed within the CD3ζ domain,
wherein in a third ITAM region, a first motif, YxxL, is substituted with YyyL; and a second motif, YxxL, is substituted with YxxQ.

8. The polypeptide of claim 4, which comprises three immunoreceptor tyrosine-based activation motifs (ITAMs) within the CD3ζ domain,
wherein a third ITAM region is mutated to a sequence of SEQ ID NO: 31.

9. The polypeptide of claim 5, which comprises three immunoreceptor tyrosine-based activation motifs (ITAMs) within the CD3ζ domain,
wherein a third ITAM region is mutated to a sequence of SEQ ID NO: 32.

10. The polypeptide of claim 4, wherein the CD3ζ domain is a sequence represented by SEQ ID NO: 18.

11. The polypeptide of claim 4, wherein a third ITAM region is a sequence between the 91^{st} and the 113^{th} positions in a sequence represented by SEQ ID NO: 18.

12. The polypeptide of claim 4, wherein in a third ITAM region, a first motif, YxxL, is substituted with YxxQ; and a second motif, YxxLHM, is substituted with YxYVTM, wherein x is any amino acid.

13. The polypeptide of claim 5, wherein in a third ITAM region, a first motif, YxxL, is substituted with YyyL; and a second motif, YxxL, is substituted with YxxQ, wherein x and y are each any amino acid.

14. The polypeptide of claim 2, which further comprises a cytokine.

15. The polypeptide of claim 14, wherein the cytokine is IL-21.

16. The polypeptide of claim 15, wherein the cytokine is linked to a chimeric antigen receptor by a self-cleaving peptide.

17. The polypeptide of claim 14, wherein the cytokine expressed in a T cell is separated from the chimeric antigen receptor and then released to the outside of the T cell.

18. The polypeptide of claim 2, which further comprises a TGF-βR2 exodomain and an IL18R endodomain, wherein an IL18R transmembrane domain is comprised between the TGF-βR2 exodomain and the IL18R endodomain.

19. The polypeptide of claim 18, wherein the TGF-βR2 exodomain is linked to a chimeric antigen receptor by a self-cleaving peptide.

20. The polypeptide of claim 19, wherein the TGF-βR2 exodomain, IL18R transmembrane domain, and IL18R endodomain, which is expressed in a T cell, is separated from the chimeric antigen receptor, and among these, the TGF-βR2 exodomain is exposed to the outside of the T cell,
wherein the IL18R endodomain is activated by the linking of the TGF-β present outside of the T cell to the TGF-βR2 exodomain.

21. The polypeptide of claim 18, which further comprises a cytokine.

22. The polypeptide of claim 21, wherein the cytokine is IL-21.

23. The polypeptide of claim 21, wherein the cytokine is linked to an IL18R endodomain by a self-cleaving peptide.

24. The polypeptide of claim 23, wherein the cytokine expressed in a T cell is separated from the IL18R endodomain and then released to the outside of the T cell.

25. The polypeptide of claim 2, wherein the antigen binding domain binds to an antigen selected from the group consisting of IL13Rα2, an antigen associated with an angiogenesis activity, EGFRvIII, EphA2, αVβ3, mesothelin, and glypican.

26. The polypeptide of claim 1, wherein the costimulatory domain comprises one or more selected from the group consisting of 4-1BB and a CD28 domain.

27. A polypeptide represented by any one sequence of SEQ ID NOS: 23 to 30 and 34 to 37.

28. The polypeptide of claim 1 for the treatment of solid cancer.

29. A CAR-T cell, wherein a polypeptide comprising the chimeric antigen receptor according to claim 1 is expressed.

30. A CAR-T cell, wherein a polypeptide comprising the chimeric antigen receptor according to claim 27 is expressed.

31. A CAR expression vector, which is a vector expressing a chimeric antigen receptor (CAR) and which comprises an antigen binding domain; a hinge region; a transmembrane domain; a costimulatory domain; and a cytoplasmic signaling domain, wherein the CAR expression vector comprises a nucleic acid encoding a CAR,
wherein the nucleic acid encoding a CAR comprises a nucleic acid encoding the costimulatory domain and a nucleic acid encoding a CD3ζ domain as the signaling domain, and which further comprises a nucleic acid encoding IL-7Rα or a part thereof that is interposed between the nucleic acid encoding the costimulatory domain and the nucleic acid encoding the CD3ζ domain.

32. A CAR expression vector, which is a vector expressing a chimeric antigen receptor (CAR) and which comprises an antigen binding domain; a hinge region; a transmembrane domain; a costimulatory domain; and a cytoplasmic signaling domain, wherein the CAR expression vector comprises a nucleic acid encoding a CAR,
wherein the nucleic acid encoding a CAR comprises a nucleic acid encoding the costimulatory domain and a nucleic acid encoding a CD3ζ domain as the signaling domain, and which further comprises a nucleic acid encoding IL-2Rβ or a part thereof that is interposed between the nucleic acid encoding the costimulatory domain and the nucleic acid encoding the CD3ζ domain.

33. The CAR expression vector of claim 31, wherein a part of IL-7Rα is a sequence of SEQ ID NO: 15.

34. The CAR expression vector of claim 32, wherein a part of IL-2Rβ is a sequence of SEQ ID NO: 17.

35. The CAR expression vector of claim 31, wherein the nucleic acid encoding the CD3ζ domain is a nucleic acid encoding a CD3ζ domain, in which in a third ITAM region among the three ITAMs present within the CD3ζ domain, a first motif, YxxL, and a second motif, YxxLHM, are substituted.

36. The CAR expression vector of claim 32, wherein the nucleic acid encoding the CD3ζ domain is a nucleic acid encoding a CD3ζ domain, in which in a third ITAM region among the three ITAMs present within the CD3ζ domain, a first motif, YxxL, and a second motif, YxxL, are substituted.

37. The CAR expression vector of claim 35, wherein the nucleic acid encoding the CD3ζ domain is a nucleic acid encoding a CD3ζ domain, in which in a third ITAM region, a first motif, YxxL, is substituted with YxxQ; and a second motif region, YxxLHM, is substituted with YxYVTM, wherein x is any amino acid.

38. The CAR expression vector of claim 36, wherein the nucleic acid encoding the CD3ζ domain is a nucleic acid encoding a CD3ζ domain, in which in a third ITAM region, a first motif, YxxL, is substituted with YyyL; and a second motif, YxxL, is substituted with YxxQ, wherein x and y are each any amino acid.

39. The CAR expression vector of claim 31, which further comprises a nucleic acid encoding cytokine IL-21.

40. The CAR expression vector of claim 39, wherein the nucleic acid encoding cytokine IL-21 is linked to a nucleic acid encoding a CAR through a nucleic acid encoding a self-cleaving peptide.

41. The CAR expression vector of claim 31, which further comprises a nucleic acid encoding a TGF-βR2 exodomain and a nucleic acid encoding an IL18R transmembrane domain and an IL18R endodomain.

42. The CAR expression vector of claim 41, wherein the nucleic acid encoding a TGF-βR2 exodomain is linked to a nucleic acid encoding a CAR through a nucleic acid encoding a self-cleaving peptide.

43. The CAR expression vector of claim 41, which further comprises a nucleic acid encoding cytokine IL-21.

44. The CAR expression vector of claim 43, wherein the nucleic acid encoding cytokine IL-21 is linked to a nucleic acid encoding IL18R endodomain through a self-cleaving peptide-encoding nucleic acid.

45. A CAR-T cell, which is prepared by introducing the vector according to claim 31.

46. A CAR-T cell, which is prepared by introducing the vector according to claim 41.

47. A CAR-T cell, which is prepared by introducing the vector according to claim 43.

48. An anticancer agent comprising the CAR-T cell according to claim 45.

49. The CAR expression vector of claim 37, wherein the costimulatory domain is one or more selected from the group consisting of 4-1BB and a CD28 domain.


SEQUENCE LISTING (43 sequences)

SEQ ID NO: 1 - protein, 112 aa, Artificial Sequence
  antigen binding wild type IL13 domain binding to IL13Ra2
GPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 2 - protein, 92 aa, Artificial Sequence
  antigen binding domain capable of binding to an antigen associated with an angiogenic activity
EVVAATPTSLLISWRHPHFPTRYYRITYGETGGNSPVQEFTVLQPPSTATISGLKPGVDYTITVYAVVERNGRELNTPPISINYRTHHHHHH

SEQ ID NO: 3 - protein, 252 aa, Artificial Sequence
  antigen binding domain binding to EGFRvIII
QVQLQESGGGLVKPGGSLKLSCAASGFTFSKFGMSWVRQTPDKRLEWVASISTGGYNTFYSDNVKGRFTISRDNAKNTLYLQMSSLKSEDTAMYYCARGYSSTSFAMDYWGQGTMVTVSSGSTSGSGKPGSGEGSDIQMTQSPSSLSASVGDRVTITCMTSTDIDDDMNWYQQKPGKTPKLLIYEGNTLRPGVPSRFSGSGSGTDFIFTISSLQPEDIATYYCLQSFNVPLTFGGGTKVEIKEQKLISEEDL

SEQ ID NO: 4 - protein, 141 aa, Artificial Sequence
  antigen binding domain binding to EphA2
DRHTVFWNSSNPKFRNEDYTIHVQLNDYVDIICPHYEDHSVADAAMEQYILYLVEHEEYQLCQPQSKDQVRWQCNRPSAKHGPEKLSEKFQRFTAFALAKEFKAGHSYYYISKPIHQHEDRCLRLKVTVSGEQKLISEEDL

SEQ ID NO: 5 - protein, 104 aa, Artificial Sequence
  antigen binding domain binding to alphaVbeta3
VSDVPRDLEVVAATPTSLLISWDAPAVTVRYYRITYGETGGNSPVQEFTVPGSKSTATISGLKPGVDYTITVYAVTPRGDWNEGSKPISINYRTEQKLISEEDL

SEQ ID NO: 6 - protein, 418 aa, Artificial Sequence
  antigen binding domain binding to glypican one
TSPCDNFDCQNGAQCIVRINEPICQCLPGYQGEKCEKLVSVNFINKESYLQIPSAKVRPQTNITLQIATDEDSGILLYKGDKDHIAVELYRGRVRASYDTGSHPASAIYSVETINDGNFHIVELLALDQSLSLSVDGGNPKIITNLSKQSTLNFDSPLYVGGMPGKSNVASLRQAPGQNGTSFHGCIRNLYINSELQDFQKVPMQTGILPGCEPCHKKVCAHGTCQPSSQAGFTCECQEGWMGPLCDQRTNDPCLGNKCVHGTCLPINAFSYSCKCLEGHGGVLCDEEEDLFNPCQAIKCKHGKCRLSGLGQPYCECSSGYTGDSCDREISCRGERIRDYYQKQQGYAACQTTKKVSRLECRGGCAGGQCCGPLRSKRRKYSFECTDGSSFVDEVEKVVKCGCTRCVSEQKLISEEDL

SEQ ID NO: 7 - protein, 262 aa, Artificial Sequence
  antigen binding domain binding to mesothelin
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSGSTSGSGKPGSGEGSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGS

SEQ ID NO: 8 - protein, 47 aa, Artificial Sequence
  hinge region sequence 1
KPTTTPAPRPPTPAPTIASQPLSLRPEACRPAAGGAVHTRGLDFACD

SEQ ID NO: 9 - protein, 45 aa, Artificial Sequence
  hinge region sequence 2
KPTTTPAPRPPTPAPTIASQPLSLRPEAARPAAGGAVHTRGLDFA

SEQ ID NO: 10 - protein, 21 aa, Artificial Sequence
  transmembrane domain sequence 1
IYIWAPLAGTCGVLLLSLVIT

SEQ ID NO: 11 - protein, 23 aa, Artificial Sequence
  transmembrane domain sequence 2
LAYLLDGILFIYGVILTALFLRV

SEQ ID NO: 12 - protein, 42 aa, Artificial Sequence
  4-1BB
KRGRKKLLYIFKQPFMRPVQTTQEEDGCSCRFPEEEEGGCEL

SEQ ID NO: 13 - protein, 41 aa, Artificial Sequence
  wild type CD28
RSKRSRLLHSDYMNMTPRRPGPTRKHYQPYAPPRDFAAYRS

SEQ ID NO: 14 - protein, 459 aa, Artificial Sequence
  IL7Ra
MTILGTTFGMVFSLLQVVSGESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 15 - protein, 64 aa, Artificial Sequence
  part of IL7Ra
KKRIKPIVWPSLPDHKKTLEHLCKKPRKNLNVSFNPESFLDCQIHRVDDIQARDEVEGFLQDTF

SEQ ID NO: 16 - protein, 551 aa, Artificial Sequence
  IL2Rb
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 17 - protein, 104 aa, Artificial Sequence
  part of IL2Rb
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHL

SEQ ID NO: 18 - protein, 113 aa, Artificial Sequence
  CD3zeta
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 19 - protein, 137 aa, Artificial Sequence
  TGF bR2 exodomain
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 20 - protein, 219 aa, Artificial Sequence
  transmembrane domain and endodomain of IL18R
PGHVFTRGMIIAVLILVAVVCLVTVCVIYRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 21 - protein, 133 aa, Artificial Sequence [sequence omitted]

SEQ ID NO: 22 - protein, 359 aa, Artificial Sequence
  YYB 103 CAR
MGWSCIILFLVATATGVHSGPVPPSTALRKLIEELVNITQNQKAPLCNGSMVWSINLTAGMYCAALESLINVSGCSAIEKTQDMLDGFCPHKVSAGQFSSLHVRDTKIEVAQFVKDLLLHLKKLFKEGQFNGGGPRKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 23 - protein, 998 aa, Artificial Sequence
  polypeptide No 1
MGWSCIILFLVATATGVHSGPVPPSTALRKLIEELVNITQNQKAPLCNGSMVWSINLTAGMYCAALESLINVSGCSAIEKTQDMLDGFCPHKVSAGQFSSLHVRDTKIEVAQFVKDLLLHLKKLFKEGQFNGGGPRKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELKKRIKPIVWPSLPDHKKTLEHLCKKPRKNLNVSFNPESFLDCQIHRVDDIQARDEVEGFLQDTFRVKFSRSADAPAYQQGQNQLYNELNLGRREEYDVLDKRRGRDPEMGGKPQRRKNPQEGLYNELQKDKMAEAYSEIGMKGERRRGKGHDGLYLPQSTATKDTYDYVTMQALPPREGRGSLLTCGDVEENPGPMALPVTALLLPLALLLHAARPTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDPGHVFTRGMIIAVLILVAVVCLVTVCVIYRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSESRRKRSGSGATNFSLLKQAGDVEENPGPMYRMQLLSCIALSLALVTNSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 24 - protein, 818 aa, Artificial Sequence
  polypeptide No 2
MGWSCIILFLVATATGVHSGPVPPSTALRKLIEELVNITQNQKAPLCNGSMVWSINLTAGMYCAALESLINVSGCSAIEKTQDMLDGFCPHKVSAGQFSSLHVRDTKIEVAQFVKDLLLHLKKLFKEGQFNGGGPRKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELKKRIKPIVWPSLPDHKKTLEHLCKKPRKNLNVSFNPESFLDCQIHRVDDIQARDEVEGFLQDTFRVKFSRSADAPAYQQGQNQLYNELNLGRREEYDVLDKRRGRDPEMGGKPQRRKNPQEGLYNELQKDKMAEAYSEIGMKGERRRGKGHDGLYLPQSTATKDTYDYVTMQALPPREGRGSLLTCGDVEENPGPMALPVTALLLPLALLLHAARPTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDPGHVFTRGMIIAVLILVAVVCLVTVCVIYRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 25 - protein, 594 aa, Artificial Sequence
  polypeptide No 5
MGWSCIILFLVATATGVHSGPVPPSTALRKLIEELVNITQNQKAPLCNGSMVWSINLTAGMYCAALESLINVSGCSAIEKTQDMLDGFCPHKVSAGQFSSLHVRDTKIEVAQFVKDLLLHLKKLFKEGQFNGGGPRKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELKKRIKPIVWPSLPDHKKTLEHLCKKPRKNLNVSFNPESFLDCQIHRVDDIQARDEVEGFLQDTFRVKFSRSADAPAYQQGQNQLYNELNLGRREEYDVLDKRRGRDPEMGGKPQRRKNPQEGLYNELQKDKMAEAYSEIGMKGERRRGKGHDGLYLPQSTATKDTYDYVTMQALPPREGRGSLLTCGDVEENPGPMYRMQLLSCIALSLALVTNSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 26 - protein, 423 aa, Artificial Sequence
  polypeptide No 6
MGWSCIILFLVATATGVHSGPVPPSTALRKLIEELVNITQNQKAPLCNGSMVWSINLTAGMYCAALESLINVSGCSAIEKTQDMLDGFCPHKVSAGQFSSLHVRDTKIEVAQFVKDLLLHLKKLFKEGQFNGGGPRKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELKKRIKPIVWPSLPDHKKTLEHLCKKPRKNLNVSFNPESFLDCQIHRVDDIQARDEVEGFLQDTFRVKFSRSADAPAYQQGQNQLYNELNLGRREEYDVLDKRRGRDPEMGGKPQRRKNPQEGLYNELQKDKMAEAYSEIGMKGERRRGKGHDGLYLPQSTATKDTYDYVTMQALPPR

SEQ ID NO: 27 - protein, 1038 aa, Artificial Sequence
  polypeptide No 7
MGWSCIILFLVATATGVHSGPVPPSTALRKLIEELVNITQNQKAPLCNGSMVWSINLTAGMYCAALESLINVSGCSAIEKTQDMLDGFCPHKVSAGQFSSLHVRDTKIEVAQFVKDLLLHLKKLFKEGQFNGGGPRKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLRVKFSRSADAPAYQQGQNQLYNELNLGRREEYDVLDKRRGRDPEMGGKPQRRKNPQEGLYNELQKDKMAEAYSEIGMKGERRRGKGHDGLYLSLSTATKDTYLPQHMQALPPREGRGSLLTCGDVEENPGPMALPVTALLLPLALLLHAARPTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDPGHVFTRGMIIAVLILVAVVCLVTVCVIYRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSESRRKRSGSGATNFSLLKQAGDVEENPGPMYRMQLLSCIALSLALVTNSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 28 - protein, 858 aa, Artificial Sequence
  polypeptide No 8
MGWSCIILFLVATATGVHSGPVPPSTALRKLIEELVNITQNQKAPLCNGSMVWSINLTAGMYCAALESLINVSGCSAIEKTQDMLDGFCPHKVSAGQFSSLHVRDTKIEVAQFVKDLLLHLKKLFKEGQFNGGGPRKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLRVKFSRSADAPAYQQGQNQLYNELNLGRREEYDVLDKRRGRDPEMGGKPQRRKNPQEGLYNELQKDKMAEAYSEIGMKGERRRGKGHDGLYLSLSTATKDTYLPQHMQALPPREGRGSLLTCGDVEENPGPMALPVTALLLPLALLLHAARPTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDPGHVFTRGMIIAVLILVAVVCLVTVCVIYRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 29 - protein, 634 aa, Artificial Sequence
  polypeptide No 11
MGWSCIILFLVATATGVHSGPVPPSTALRKLIEELVNITQNQKAPLCNGSMVWSINLTAGMYCAALESLINVSGCSAIEKTQDMLDGFCPHKVSAGQFSSLHVRDTKIEVAQFVKDLLLHLKKLFKEGQFNGGGPRKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLRVKFSRSADAPAYQQGQNQLYNELNLGRREEYDVLDKRRGRDPEMGGKPQRRKNPQEGLYNELQKDKMAEAYSEIGMKGERRRGKGHDGLYLSLSTATKDTYLPQHMQALPPREGRGSLLTCGDVEENPGPMYRMQLLSCIALSLALVTNSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 30 - protein, 463 aa, Artificial Sequence
  polypeptide No 12
MGWSCIILFLVATATGVHSGPVPPSTALRKLIEELVNITQNQKAPLCNGSMVWSINLTAGMYCAALESLINVSGCSAIEKTQDMLDGFCPHKVSAGQFSSLHVRDTKIEVAQFVKDLLLHLKKLFKEGQFNGGGPRKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLRVKFSRSADAPAYQQGQNQLYNELNLGRREEYDVLDKRRGRDPEMGGKPQRRKNPQEGLYNELQKDKMAEAYSEIGMKGERRRGKGHDGLYLSLSTATKDTYLPQHMQALPPR

SEQ ID NO: 31 - protein, 23 aa, Artificial Sequence
  mutated 3rd ITAM 1
YLPQSTATKDTYDYVTMQALPPR

SEQ ID NO: 32 - protein, 23 aa, Artificial Sequence
  mutated 3rd ITAM 2
YLSLSTATKDTYLPQHMQALPPR

SEQ ID NO: 33 - protein, 252 aa, Artificial Sequence
  antigen binding domain binding to EGFRvIII
QVQLQESGGGLVKPGGSLKLSCAASGFTFSKFGMSWVRQTPDKRLEWVASIDPENDETFYSDNVKGRFTISRDNAKNTLYLQMSSLKSEDTAMYYCARGYGSTSFAMDYWGQGTMVTVSSGSTSGSGKPGSGEGSDIQMTQSPSSLSASVGDRVTITCMTSTDIDDDMNWYQQKPGKTPKLLIYEGNTLRPGVPSRFSGSGSGTDFIFTISSLQPEDIATYYCLQSFNGPLTFGGGTKVEIKEQKLISEEDL

SEQ ID NO: 34 - protein, 499 aa, Artificial Sequence
  polypeptide No 13
MALPVTALLLPLALLLHAARPQVQLQESGGGLVKPGGSLKLSCAASGFTFSKFGMSWVRQTPDKRLEWVASISTGGYNTFYSDNVKGRFTISRDNAKNTLYLQMSSLKSEDTAMYYCARGYSSTSFAMDYWGQGTMVTVSSGSTSGSGKPGSGEGSDIQMTQSPSSLSASVGDRVTITCMTSTDIDDDMNWYQQKPGKTPKLLIYEGNTLRPGVPSRFSGSGSGTDFIFTISSLQPEDIATYYCLQSFNVPLTFGGGTKVEIKEQKLISEEDLGGGPRKPTTTPAPRPPTPAPTIASQPLSLRPEAARPAAGGAVHTRGLDFALAYLLDGILFIYGVILTALFLRVKRGRKKLLYIFKQPFMRPVQTTQEEDGCSCRFPEEEEGGCELKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 35 - protein, 499 aa, Artificial Sequence
  polypeptide No 14
MALPVTALLLPLALLLHAARPQVQLQESGGGLVKPGGSLKLSCAASGFTFSKFGMSWVRQTPDKRLEWVASIDPENDETFYSDNVKGRFTISRDNAKNTLYLQMSSLKSEDTAMYYCARGYGSTSFAMDYWGQGTMVTVSSGSTSGSGKPGSGEGSDIQMTQSPSSLSASVGDRVTITCMTSTDIDDDMNWYQQKPGKTPKLLIYEGNTLRPGVPSRFSGSGSGTDFIFTISSLQPEDIATYYCLQSFNGPLTFGGGTKVEIKEQKLISEEDLGGGPRKPTTTPAPRPPTPAPTIASQPLSLRPEAARPAAGGAVHTRGLDFALAYLLDGILFIYGVILTALFLRVKRGRKKLLYIFKQPFMRPVQTTQEEDGCSCRFPEEEEGGCELKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 36 - protein, 349 aa, Artificial Sequence
  polypeptide No 15
MGVKVLFALICIAVAEAVSDVPRDLEVVAATPTSLLISWDAPAVTVRYYRITYGETGGNSPVQEFTVPGSKSTATISGLKPGVDYTITVYAVTPRGDWNEGSKPISINYRTEQKLISEEDLGGGPRKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 37 - protein, 353 aa, Artificial Sequence
  polypeptide No 16
MALPVTALLLPLALLLHAARPVSDVPRDLEVVAATPTSLLISWDAPAVTVRYYRITYGETGGNSPVQEFTVPGSKSTATISGLKPGVDYTITVYAVTPRGDWNEGSKPISINYRTEQKLISEEDLGGGPRKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 38 - protein, 351 aa, Artificial Sequence
  YYB 107 CAR
MGWSCIILFLVATATGVHSVSDVPRDLEVVAATPTSLLISWDAPAVTVRYYRITYGETGGNSPVQEFTVPGSKSTATISGLKPGVDYTITVYAVTPRGDWNEGSKPISINYRTEQKLISEEDLGGGPRKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 39 - protein, 497 aa, Artificial Sequence
  YYB 105 CAR
MGWSCIILFLVATATGVHSQVQLQESGGGLVKPGGSLKLSCAASGFTFSKFGMSWVRQTPDKRLEWVASISTGGYNTFYSDNVKGRFTISRDNAKNTLYLQMSSLKSEDTAMYYCARGYSSTSFAMDYWGQGTMVTVSSGSTSGSGKPGSGEGSDIQMTQSPSSLSASVGDRVTITCMTSTDIDDDMNWYQQKPGKTPKLLIYEGNTLRPGVPSRFSGSGSGTDFIFTISSLQPEDIATYYCLQSFNVPLTFGGGTKVEIKEQKLISEEDLGGGPRKPTTTPAPRPPTPAPTIASQPLSLRPEAARPAAGGAVHTRGLDFALAYLLDGILFIYGVILTALFLRVKRGRKKLLYIFKQPFMRPVQTTQEEDGCSCRFPEEEEGGCELKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 40 - protein, 21 aa, Artificial Sequence
  T2A peptide
GSGEGRGSLLTCGDVEENPGP

SEQ ID NO: 41 - protein, 22 aa, Artificial Sequence
  P2A peptide
GSGATNFSLLKQAGDVEENPGP

SEQ ID NO: 42 - protein, 23 aa, Artificial Sequence
  E2A peptide
GSGQCTNYALLKLAGDVESNPGP

SEQ ID NO: 43 - protein, 25 aa, Artificial Sequence
  F2A peptide
GSGVKQTLNFDLLKLAGDVESNPGP